SEQUENCE LISTING (22 sequences)

SEQ ID NO: 1 - DNA, 1711 bp, Homo sapiens
  misc_feature (1645)..(1645): n is a, c, g, or t
cattgtgttggggcccaggaagcctccgcctcagagatgctaccgctgctgctgcccctgctgtgggcaggggccctggctcaggagcggagattccagctggaggggccagagtcactgacggtgcaggagggtctgtgcgtcctcgtaccctgcagattgcccactacccttccagcctcgtactatggttatggctactggttcctggaaggggctgatgttccagtggccacaaacgacccagacgaagaagtgcaggaggagacccggggccgattccacctcctctgggatcccagaaggaagaactgctccctgagcatcagagatgcccggaggagggacaatgctgcatacttctttcggttgaagtccaaatggatgaaatacggttatacatcttccaagatatatgtgcgtgtgatggccctgacccacaggcccaacatctccatcccaggacctggagtctggccatccagcaatctgacctgctctgtgccctgggtctgtgagcaggggacgccccccatcttctcctggatgtcagctgccccccacctcctgggccccaggaccacccagtcctcggtgctcacaatcaccccggcccaggaccacagcaccaacctcacctgtcaggtgacgttccctggagccggtgtgaccatggagagaaccatccagctcaatgtctcctatgctccacagaaagtggccatcagcatcttccaaggaaacagcgcagccttcaaaatcctgcaaaacacctcgtccctccctgtcctggagggccaggctctgcggctgctctgtgatgctgacggcaacccccctgcacacctgagctggttccagggcttccccgccctgaacgccacccccatctccaataccggggtcctggagctgcctcaagtagggtctgcagaagaaggagatttcacctgccgtgctcagcatcctctgggctccctgcaaatctctctgagtctctttgtgcattggaaaccagaaggcagggctggtggtgtcctgggagcagtctggggagctagcatcacaaccctggttttcctctgtgtttgcttcatcttcagagtgaagactagaaggaagaaagcagcccagccagtgcaaaacacggatgatgtgaaccccgtcatggtctcaggctccaggggtcatcagcaccagttccagacaggcatagtttcagaccaccctgctgaggctggccccatctcagaagatgagcaggagctccactacgctgtcctacacttccacaaggtgcaacctcaggaaccaaaggtcaccgacactgagtactcagaaatcaagatacacaagtgaggaattgtccaaagcataaccttgattggagagacatgggtacctctcagtgtattggttactagggctgccacagcaatgtaccacaaaccgagtgacataaacacagaactttattttcgatagtttcagatgttagaggtctgagaacaaggtgttatcagggttggtcccttctaaggcctctcttgttggctttgagatggctgtctcctccttgtgtcttcacatggtctttcctctgagtgtgtttgtgtcctaatcttctcttcttataaagacactnagtcatattggattagggcctccccatgacctaatttaaataaattaactatttaaagaccctcca

SEQ ID NO: 2 - protein, 440 aa, Homo sapiens
MLPLLLPLLWAGALAQERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKIYVRVMALTHRPNISIPGPGVWPSSNLTCSVPWVCEQGTPPIFSWMSAAPHLLGPRTTQSSVLTITPAQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 3 - DNA, 20 bp, ARTIFICIAL
  OLIGONUCLEOTIDE PRIMER FOR SIGLEC-6
tggagctgcctcaagtaggg

SEQ ID NO: 4 - DNA, 20 bp, ARTIFICIAL
  OLIGONUCLEOTIDE PRIMER FOR SIGLEC-6
cgcggcaggtgaaatctcct

SEQ ID NO: 5 - DNA, 23 bp, ARTIFICIAL
  OLIGONUCLEOTIDE PRIMER FOR SIGLEC-6
caccatgctaccgctgctgctga

SEQ ID NO: 6 - DNA, 23 bp, ARTIFICIAL
  OLIGONUCLEOTIDE PRIMER FOR SIGLEC-6
tcacttgtgtatcttgatttctg

SEQ ID NO: 7 - DNA, 333 bp, Mus musculus
gacattgtgctgacccaatctccagcttctttggctgtgtctctagggcagagggccaccatctcctgcaaggccagccaaaatgttgattatgatggtgacagttatatgaactggtaccaacagaaaccagggcagccacccaaactcctcatctatgctgcgtccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 8 - protein, 111 aa, Mus musculus
DIVLTQSPASLAVSLGQRATISCKASQNVDYDGDSYMNWYQQKPGQPPKLLIYAASNLESGIPARFSGSGSGTDFTLNIHPVEEEDAATYYCQQSNEDPWTFGGGTKLEIK

SEQ ID NO: 9 - DNA, 345 bp, Mus musculus
cagctgcagcagtctggacctgagctggtgaggcctgggacttcagtgaagatttcctgcaaggcttctgcctataccttcctcacctactacatgaactgggtgaagcagaggcctggacagggccttgagtggattggacagatttttcctgcaagtggtagtactaactacaatgagatgttcaagggcaaggccacattgactgtagacacatcctccagcacagcctacatactgctaaacagcctgacatctgaggactctgcggtctatttctgtacaagatctttcgggggggggtttgcttactggggccaagggactctggtcactgtctctgca

SEQ ID NO: 10 - protein, 117 aa, Mus musculus
QVQLQQSGPELVRPGTSVKISCKASAYTFLTYYMNWVKQRPGQGLEWIGQIFPASGSTNYNEMFKGKATLTVDTSSSTAYILLNSLTSEDSAVYFCTRSFGGGFAYWGQGTLVTVSA

SEQ ID NO: 11 - DNA, 45 bp, Artificial
  Nucleic Acid Sequence of Light Chain CDR1 of MAb 239-90
aaggccagccaaaatgttgattatgatggtgacagttatatgaac

SEQ ID NO: 12 - protein, 15 aa, Artificial
  Amino Acid Sequence of Light Chain CDR1 of MAb 239-90
KASQNVDYDGDSYMN

SEQ ID NO: 13 - DNA, 21 bp, Artificial
  Nucleic Acid Sequence of Light Chain CDR2 of MAb 239-90
gctgcgtccaatctagaatct

SEQ ID NO: 14 - protein, 7 aa, Artificial
  Amino Acid Sequence of Light Chain CDR2 of MAb 239-90
AASNLES

SEQ ID NO: 15 - DNA, 27 bp, Artificial
  Nucleic Acid Sequence of Light Chain CDR3 of MAb 239-90
cagcaaagtaatgaggatccgtggacg

SEQ ID NO: 16 - protein, 9 aa, Artificial
  Amino Acid Sequence of Light Chain CDR3 of MAb 239-90
QQSNEDPWT

SEQ ID NO: 17 - DNA, 30 bp, Artificial
  Nucleic Acid Sequence of Heavy Chain CDR1 of MAb 239-90
gcctataccttcctcacctactacatgaac

SEQ ID NO: 18 - protein, 10 aa, Artificial
  Amino Acid Sequence of Heavy Chain CDR1 of MAb 239-90
AYTFLTYYMN

SEQ ID NO: 19 - DNA, 51 bp, Artificial
  Nucleic Acid Sequence of Heavy Chain CDR2 of MAb 239-90
cagatttttcctgcaagtggtagtactaactacaatgagatgttcaagggc

SEQ ID NO: 20 - protein, 17 aa, Artificial
  Amino Acid Sequence of Heavy Chain CDR2 of MAb 239-90
QIFPASGSTNYNEMFKG

SEQ ID NO: 21 - DNA, 24 bp, Artificial
  Nucleic Acid Sequence of Heavy Chain CDR3 of MAb 239-90
tctttcgggggggggtttgcttac

SEQ ID NO: 22 - protein, 8 aa, Artificial
  Amino Acid Sequence of Heavy Chain CDR3 of MAb 239-90
SFGGGFAY

(19) United States
(12) Patent Application Publication
(10) Pub. No.: US 2008/0267973 A1
(43) Pub. Date: Oct. 30, 2008

(54) Diagnosis and Treatment of Siglec-6 Associated Diseases

(75) Inventors: Shen-Wu Wang, Newbury, CA (US); Guanghu Hu, Phoenixville, PA (US); Zhengbin Yao, Sugarland, TX (US)

Correspondence Address: Nikolaos C. George;JONES DAY, 222 E. 41st. Street, New York, NY, 10017-6702, US

(73) Assignee: Genetech, Inc.

(21) Appl. No.: 11/629,047

(22) PCT Filed: Jun. 9, 2005
(86) PCT No.: PCT/US2005/020271

§ 371 Date: Dec. 8, 2006

Related U.S. Application Data
(60) Provisional application No. 60/578,194, filed on Jun. 9, 2004.
(60) Provisional application No. 60/580,422, filed on Jun. 17, 2004.

(51) Int. Cl. A61K 39/395; C07K 16/28; C07H 21/04; A61P 11/06; C12Q 1/02; G01N 33/566; C12Q 1/68; G01N 33/567; C12N 5/06

(52) U.S. Cl. 424/153.1; 530/389.6; 530/387.3; 536/23.53; 435/325; 424/130.1; 435/7.21; 435/6; 435/29; 436/501

(57) ABSTRACT

The present invention relates to agonists, antagonists, and other molecules that specifically bind SIGLEC-6 on mast cells, their use in the treatment of asthma and other SIGLEC-6 mediated diseases or disorders, methods of diagnosing such diseases or disorders, and methods of screening for candidate compounds capable of modulating SIGLEC-6 activity in mast cells. The present invention also relates to the diagnosis and treatment of B-cell related disorders using compounds that bind and/or modulate SIGLEC-6.

### CROSS REFERENCE TO RELATED APPLICATIONS

This application claims priority to U.S. Provisional Application No. 60/578,194, which was filed on 9 Jun. 2004 and U.S. Provisional Application 60/580,422, which was filed on 17 Jun. 2004, both of which are hereby incorporated by reference in their entirety.

### FIELD OF THE INVENTION

The present invention relates to Siglec-6, which is highly expressed on the surface of mast cells and circulating blood monocytes (CBMCs) and it use in the treatment of mast cell related diseases or disorders. It also relates to the use of Siglec-6 in the diagnosis and treatment of B-cell related diseases and disorders, such as leukemia and B-cell lymphoma.

### BACKGROUND OF THE INVENTION

A group of sialic acid-dependent adhesion molecules has been described within the superfamily of immunoglobulin-like molecules (Kelm, S. et al., 1998 Eur. J. Biochem 255:663-672). The term “Siglec” has been adopted to describe this family (Sialic acid-binding Ig-related lectins). To date, the members of the group include Siglec-1 (sialoadhesin), Siglec-2 (CD22), Siglec-3 (CD33), Siglec-4 (myelin-associated glycoprotein or MAG), Siglec-4-b (Schwann cell myelin protein or SMP), Siglec-5 (OB-BP2), Siglec-6 (OB-BP1, CD33L), Siglec-7, Siglec-8, Siglec-9, Siglec-10, Siglec-11, and Siglec 12.

The biological activity of the protein members of the Siglec group is thought to be involved in diverse biological processes such as hemopoiesis, neuronal development and immunity (Vinson, M. et al., 1996 supra). Studies also suggest that these proteins mediate cell adhesion/cell signaling through recognition of sialyated cell surface glycans (Kelm, S. et al., 1996 Glycoconj. J. 13:913-926; Kelm, S. et al., 1998 Eur. J. Biochem. 255:663-672; Vinson, M. et al., 1996 J. Biol. Chem. 271:9267-9272).

The known Siglec proteins are expressed in diverse hemopoietic cell types, yet they all share a similar structure including a single N-terminal V-set domain (membrane-distal) followed by variable numbers of extracellular C2-set domains, a transmembrane domain, and a short cytoplasmic tail. Additionally, the terminal V-set domain has an unusual intrasheet disulfide bridge that is unique among members of the Ig superfamily (Williams, A. F. and Barclay, A. N. 1988 Annu. Rev. Immunol. 6:381-405; Williams, A. F., et al., 1989 Cold Spring Harbor Symp. Quant. Biol 54:637-647; Pedraza, L., et al., 1990 J. Cell. Biol. 111:2651-2661).

Results of various research approaches, including truncating mutants (Nath, D., et al., J. Biol. Chem. 270:26184-26191), site-directed mutagenesis (Vinson, M., et al., 1996 J. Biol. Chem. 271:9267-9272; Van der Merwe, P. a., et al., 1996 J. Biol. Chem. 271:9273-9280), X-ray crystallography and NMR (discussed in: Crocker, P. R., et al., 1997 Glycoconjugate J. 14:601-609) have demonstrated that the GFCC′C″ face of the N-terminal V-set domain of known Siglec proteins interact with sialic acid. Thus, the V-set domain mediates cell-to-cell adhesion by interacting with sialic acid.

The purported ligands for the known Siglec proteins are glycoproteins or glycolipids on other cells, or in some instances on the same cell, modified to include sugars or sialic acid. There are approximately 40 naturally occurring sialic acids (Sia) adding to the structural diversity of cell surface glycoproteins. The most common are NeuSAc, Neu9Ac2 and Neu5Gc, occurring in terminal positions linked to other sugars like Gal, GalNAc, GlcNAc and Sia itself on glycoproteins and glycolipids. It is postulated that the pattern of expression of sialic acids in certain cell types is controlled by specific expression of sialyltransferases (Paulson, J. C. et al., 1989 J. Biol. Chem. 264:10931-10934). The Siglec proteins may recognize not only the terminal sialic acids but also the context of these moieties based on pre-terminal sugars to which they are attached (Kelm, S., et al., 1996 Glycoconj. J. 13:913-926).

Siglecs may mediate cell to cell adhesion by functioning as sialic acid-dependent lectins with distinct specificities for the type of sialic acid and its linkage to subterminal sugars (Kelm, S., 1994 supra; Powell, L. D., et al., 1994 J. Biol. Chem. 269:10628-10636; Sjoberg, E., et al., 1994 J. Cell Biol. 126:549-562; Collins, B., E., et al., 1997 J. Biol. Chem. 272:1248-1255). The structural interactions between sialoadhesin and carbohydrates have been analyzed (for example, see Collins et al., J Biol Chem 272:16889-95, 1997; see also May et al., Mol. Cell 1:719-28, 1998). Siglecs exhibit functional protein-carbohydrate recognition through specific siaylated glycoconjugates on their cognate molecules, and some of them bind with glycans that terminate in α-2,3 linked sialic acids (Kelm et al., Curr. Biol. 4:965-72, 1994). The sialic acid-binding activity usually resides on the N-terminal V-set Ig-like domain, and may also involve the penultimate Ig-like domain. Some members of this group are reported to exhibit distinct specificities for both the type of sialic acid and its linkage to subterminal sugars.

The amino acid sequences of the cytoplasmic tails of several Siglec proteins strongly suggest that they participate in intracellular signaling. For example, Siglec-2 has 6 tyrosines in the cytoplasmic domain, two of which reside within ITAM (Immunotyrosine-based activation motifs) motifs which mediate activation, and four within ITIM (Immunotyrosine-based inhibition motifs) motifs which mediate inhibition (Taylor, V. et al., 1999 J. Biol. Chem. 274:11505-11512). Phosphorylation of the ITAM motif tyrosines would allow recruitment of Src, whereas phosphorylation of ITIM motif tyrosines would allow recruitment of SHP-1 and SHP-2. Siglec-3 contains two ITIMs that recruit SHP-1 and SHP-2 upon phosphorylation (Taylor, V. et al., 1999 supra). Siglec-6 also has putative SLAM-like signaling motifs in the cytoplasmic tail; SLAM is an acronym for Signaling Lymphocyte Activation Molecule. (Patel, N. et al., 1999 J. Biol. Chem. 274:22729-22738).

There is mounting evidence that inflammatory cell infiltrates play a significant role in driving the pathogenesis of asthma and other allergic diseases by damaging tissue and releasing pro-inflammatory agents. Activated eosinophils, neutrophils, macrophages, mast cells and lymphocytes increase in number at sites of inflammation and each are capable of modifying the overall inflammatory response (Busse, W. W. 1998 J. Allergy Clin. Immunol. 102:S17-22). Eosinophils are of particular interest in asthma and allergy due to their conspicuous appearance at the sites of allergen-driven inflammation (Kroegel, C. et al., 1994 Eur. Respir. J. 7:519-543; Haczku, A. 1998 Acta. Microbiol. Immunol. Hung. 45:19-29; Boyce, J. A. 1997 Allergy Asthma Proc. 18:293-300). Through release of toxic granule proteins, pro-inflammatory lipid mediators and cytokines, eosinophils have been implicated as major players in airway remodeling and hyperresponsiveness in asthma (Durham, S. R. 1998 Clin. Exp. Allergy 28 Suppl. 2:11-6).

Many proteins have been reported to contain a cytoplasmic inhibitory signaling motif that is associated with the transduction of inhibitory effector functions, e.g., the “immunoreceptor tyrosine-based inhibition motif,” or “ITIM” (Renard et al., Immun Rev 155:205-221, 1997). ITIMs have the consensus sequence I/VxYxxL/V and are found in the cytoplasmic portions of diverse signal transduction proteins of the immune system, many of which, like the siglecs, belong to the Ig superfamily or to the family of type II dimeric C-lectins (see Renard et al., 1997, supra). Proteins that contain ITIMs include the “killer cell Ig-like receptors,” or “KIRs,” and some members of the leukocyte Ig-like receptor or “LIR” family of proteins (Renard et al., 1997, supra; Cosman et al., Immunity 7:273-82, 1997; Borges et al., J Immunol 159:5192-96, 1997). Signal transduction by an ITIM is believed to downregulate targeted cellular activities, such as expression of cell surface proteins. Renard et al. propose that the regulation of complex cellular functions is fine-tuned by the interplay of ITIM-mediated inhibitory signal transduction and activation of the same functions by a 16-18 amino acid activitory motif, or “ITAM” sequence that is present in other proteins.

Some of the siglecs have been reported to contain one or more ITIMs in their cytoplasmic regions. CD22 has more than one ITIM and has been characterized as a negative regulator of B cell activation. CD33 and siglec 8 also are reported to contain ITIM motifs in their cytoplasmic domains (Ulyanova et al., Eur J Immunol 29:3440-49, 1999; Floyd et al., 2000, supra). An ITIM is also present in the cytoplasmic tail of p75/AIRM1/siglec 7, a protein expressed at significant levels on a subset of CD8⁺ natural killer (NK) cells (Nicoll et al., 1999, supra).

Siglec expression is restricted largely to myeloid cells of the immune system, and is believed to be involved in control of myeloid interactions, such as adhesions between antigen presenting cells (APCs), e.g., macrophages (including microglia) or dendritic cells, and other cells involved in cell-mediated immunity, such as T cells or natural killer cells. These polypeptides may function in antigen capture and uptake when expressed on APCs, and thus may provide targets for enhancing cell-based tumor vaccines. Many siglecs are observed to be expressed primarily on subsets of specific types of hematopoietic cells. CD33 expression is largely restricted to the myelomonocytic lineage, and is present on mature monocytes and tissue macrophages (Freeman et al., 1995, supra). CD22 is expressed primarily on B-cells, while siglec-8 is expressed specifically on eosinophilic granulocytes (Floyd et al., J. Biol. Chem. 275:861-866, 2000). Sialoadhesin is expressed at high levels on macrophages in chronic inflammatory conditions and in tumors, suggesting a role in host defense, and can mediate specific cell-substrate and cell-cell interactions in vitro (Crocker et al., 1994; Crocker et al., 1997, supra). Umansky et al. have reported that sialoadhesin-positive macrophages contribute to host resistance against metastasis of tumors, that these macrophages can function as antigen-presenting cells, and also that sialoadhesion expression is responsive to corticosteroids, lymphokines and cytokines (Umansky et al., 1996 and 1996).

Comparisons to other known Siglec family members (CD22, CD33, myelin-associated glycoprotein, and sialoadhesin) show that OB-BP1/Siglec-6, OB-BP2/Siglec-5, and CD33/Siglec-3 constitute a unique related subgroup with a high level of overall amino acid identity: Siglec-6 versus Siglec-5 (59%), Siglec-6 versus CD33 (63%), and Siglec-6/Siglec-5 versus CD33 (56%). The cytoplasmic domains are not as highly conserved, but display novel motifs which are putative sites of tyrosine phosphorylation, including an immunoreceptor tyrosine kinase inhibitory motif and a motif found in SLAM and SLAM-like proteins.

Siglec-6 (OB-BP1) was isolated from the TF-1 human erythroleukemic cell line (Patel, N., et al., 1999 J. Biol. Chem. 274:22729-22738). Siglec-6 is essentially the same as CD33-L1 except for a few amino acid differences. Human tissues showed high levels of Siglec-6 mRNA in placenta and moderate expression in spleen, peripheral blood leukocytes, and small intestine. A monoclonal antibody specific for Siglec-6 confirmed high expression in the cyto- and syncytiotrophoblasts of the placenta. Using this antibody on peripheral blood leukocytes showed an almost exclusive expression pattern on B cells. Recombinant forms of the extracellular domains of Siglec-6, Siglec-5, and CD33/Siglec-3 were assayed for specific binding of leptin. While Siglec-6 exhibited tight binding (K(d) 91 nM), the other two showed weak binding with K(d) values in the 1-2 microM range. Studies with sialylated ligands indicated that Siglec-6 selectively bound Neu5Acalpha2-6GalNAcalpha (sialyl-Tn) allowing its formal designation as Siglec-6. Because of Siglec-6's restricted expression pattern, it has been suggested that it may mediate cell-cell recognition events by interacting with sialylated glycoprotein ligands expressed on specific cell populations.

We have found that Siglec-6 is highly expressed on mast cells and a small sub population of B-cells, but not primary monocytes, neutrophils or T-lymphocytes. Thus, SIGLEC-6 may be beneficial as a specific and directed target for treating diseases associated with mast cell proliferation and for inhibiting mast cell mediators that lead to inflammatory or allergic diseases, including asthma. In addition, SIGLEC-6 may be beneficial as a specific and directed target for diagnosing and treating B-cell mediated diseases, such as leukemia and B-cell lymphoma.

### SUMMARY OF THE INVENTION

The present invention relates to a method of modulating an immune response induced by SIGLEC-6 expressing mast cells. The present invention includes treatment of immune diseases, including allergic diseases, such as asthma, as well as inflammatory diseases, by the use of agonists for SIGLEC-6. Because SIGLEC-6 contains an immunoreceptor tyrosine-based inhibition motif, “ITIM”, an agonist molecule that binds to SIGLEC-6, such as an antibody, would stimulate the ITIM to inhibit certain mast cell functions. This molecule may also be a bispecific molecule that crosslinks the ITIM of SIGLEC-6 with an ITAM, such as FceRI, thus inhibiting the action of the ITAM containing receptor.

The present invention also relates to a method for diagnosis and treatment of B-cell related disorders, wherein the B-cell expresses SIGLEC-6 on its cell surface. The present invention includes treatment of B-cell related diseases such as leukemia and B-cell lymphoma. Because SIGLEC-6 contains an immunoreceptor tyrosine-based inhibition motif, “ITIM”, an agonist molecule that binds to SIGLEC-6, such as an antibody, would stimulate the ITIM to inhibit B-cell functions. This molecule may also be a bispecific molecule that crosslinks the ITIM of SIGLEC-6 with an ITAM, such as FceRI, thus inhibiting the action of the ITAM containing receptor.

The present invention includes antibodies that specifically bind SIGLEC-6, including agonist antibodies, antagonist antibodies, antibodies having an Fc-mediated cellular cytotoxicity, such as antibody-dependent cell-mediated cytotoxicity (ADCC), antibody conjugates, antibodies that block binding to SIGLEC-6, and antibodies that specifically bind to SIGLEC-6 for detection and diagnostic identification of SIGLEC-6 expressing B-cells. These antibodies may be polyclonal or monoclonal and functional binding fragments thereof. Antibodies may also be single-domain antibodies having only a heavy chain or a light chain. Monoclonal antibodies may be humanized, human, chimeric, bispecific, or conjugated. The present invention also includes single chain antibodies.

Antibody conjugates may be used for the depletion of mast cells or the induction of mast cell apoptosis. Antibody conjugates may also be used for the depletion of pathological B-cells or the induction of apoptosis in SIGLEC-6 expression B-cells. The conjugated moeity may include toxins, radioactive isotopes, labels, such as photoreactive moeities, or an apoptosis inducing moeity, such as a pro-apoptotic member of the Bcl-2 family selected from Bax-α, Bak, Bcl-X_{S}, Bad, Bid, Bik, Erk, and Bok.

The present invention includes compositions of these anti-SIGLEC-6 antibodies for use in diagnosis and/or treatment. Compositions include the antibodies in combination with suitable carriers, adjuvants, diluents, excipients, and/or additives.

Another aspect of the invention relates to screening methods for identifying agents of interest that bind with (e.g., ligands) and/or modulate the biological activity of SIGLEC-6 proteins. Because SIGLEC-6 proteins are expressed in mast cells, these agents may be involved in modulating mast cells or other immune cell maturation, migration, activation, or communication with other cells. Further, SIGLEC-6 is expressed on the surface of certain B-cells and these agents may be used to deplete or kill this population of B-cells. Thus, agents that bind with and modulate the biological activity of SIGLEC-6 proteins may be effective in reducing certain symptoms of asthma and other allergic diseases, leukemia, or reduce inflammation.

The present invention also includes diagnostic methods for mast cell mediated diseases and disorders by the use of anti-SIGLEC-6 antibodies. SIGLEC-6 is highly specific for mast cells, thus one can detect the presence and frequency of mast cells in a given sample, such as a tissue biopsy, with antibodies directed to SIGLEC-6. The relative increase of SIGLEC-6 in a sample may be indicative of, e.g., asthma, in patients with increased levels of mast cells in the smooth muscle tissue of the lung. Anti-SIGLEC-6 antibodies may also be used to detect the presence or increase/decrease of cells expressing SIGLEC-6.

The present invention also includes diagnostic methods for B-cell related diseases and disorders, wherein the B-cell expresses SIGLEC-6, by the use of anti-SIGLEC-6 antibodies. SIGLEC-6 may be used to detect the presence and frequency of B-cells expressing SIGLEC-6 in a given sample, such as a blood sample from an affected patient, with antibodies directed to SIGLEC-6. The presence of SIGLEC-6 expressing B-cells in a sample may be indicative of a B-cell related disorder, such as B-cell lymphoma. Anti-SIGLEC-6 antibodies may also be used to detect the presence or increase/decrease of cells expressing SIGLEC-6.

The antibodies of the present invention may be used in a diagnostic method for detecting SIGLEC-6 expressing B-cells in tissues or body fluids. The method comprises exposing the patient sample to an antibody of the present invention and determining if the antibody binds to cells in the sample. Various diagnostic methods known in the art may be used, e.g., competitive binding assays, direct or indirect sandwich assays, and immunoprecipitation assays conducted in either heterogeneous or homogeneous stages. Expression of SIGLEC-6 protein in body fluids or tissues can be detected by immunofluorescence, FACS staining, or immunohistochemistry using an anti-SIGLEC-6 mAb.

### BRIEF DESCRIPTION OF THE FIGURES

FIG. 1 depicts the nucleic acid sequence of Siglec-6 (SEQ ID NO: 1).

FIG. 2 depicts the amino acid sequence of SIGLEC-6 protein (SEQ ID NO: 2) including the signal sequence (underlined) and the putative ITIM and SLAM regions of the 3′ region.

FIG. 3 depicts the relative expression level of Siglec-6 mRNA in various tissues and cell lines.

FIG. 4 depicts FACS staining of CBMC, LAD2 and HMC-1 illustrating SIGLEC-6 expression on the cell surface of these cells.

FIG. 5 depicts the effect of anti-SIGLEC-6 on CBMC activation via FcγRI.

FIGS. 6A and 6B shows the nucleic acid (SEQ ID NO: 7 and 9) and amino acid (SEQ ID NO: 8 and 10) sequences of light chain and heavy chain variable regions of Mab 239-90 with complementarity determining regions (CDRs) underlined.

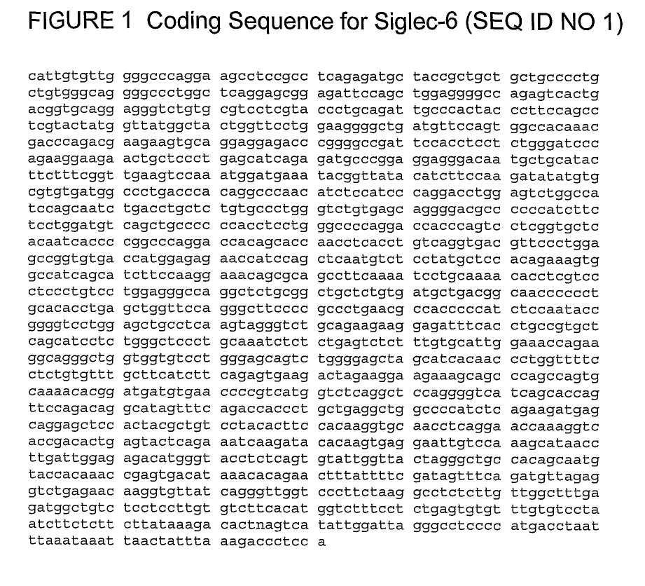

### DETAILED DESCRIPTION OF THE INVENTION

### Definitions

Terms used throughout this application are to be construed with ordinary and typical meaning to those of ordinary skill in the art. However, Applicants desire that the following terms be given the particular definition as defined below.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “sequence identity” or “sequence homology” shall be construed to mean the percentage of amino acid residues in the candidate sequence that are identical with the residue of a corresponding sequence to which it is compared, after aligning the sequences and introducing gaps, if necessary, to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software.

The phrase “substantially identical” with respect to an antibody chain polypeptide sequence may be construed as an antibody chain exhibiting at least 70%, or 80%, or 90% or 95% sequence identity to the reference polypeptide sequence.

The term “variant” when used to describe a polypeptide sequence, such as an antibody sequence, means an amino acid sequence that differs from its native counterpart by one or more amino acids, including modifications, substitutions, insertions, and deletions, but retains the same or similar biological function as its native counterpart. Variants include polypeptides having at least 70 percent sequence identity when compared to its native counterpart, at least 85 percent sequence identity, and or at least 95 percent sequence identity. Variants include, e.g., polypeptides with conservative amino acid substitutions.

The term “conservative amino acid substitution” means that an amino acid in a polypeptide has been substituted for with an amino acid having a similar side chain. For example, glycine, alanine, valine, leucine, and isoleucine have aliphatic side chains; serine and threonine have aliphatic-hydroxyl side chains; asparagine and glutamine have amide-containing side chains; phenylalanine, tyrosine, and tryptophan have aromatic side chains; lysine, arginine, and histidine have basic side chains; and cysteine and methionine have sulfur-containing side chains. Preferred conservative amino acids substitutions are valine-leucine-isoleucine, phenylalanine-tyrosine, lysine-arginine, alanine-valine, and asparagine-glutamine.

The term “fragment” when used to describe a polypeptide means an amino acid sequence subset of its native counterpart that retains any biological activity of its native counterpart. Fragments include amino acid sequences of at least 10 to 20 consecutive amino acids of the native sequence or of at least 20 to 30 consecutive amino acids of the native sequence.

The term “agonist” means any molecule that directly or indirectly promotes, enhances, or stimulates the normal function of SIGLEC-6. One type of agonist is a molecule that interacts with SIGLEC-6 in a way that mimics its ligand, including, but not limited to, an antibody or antibody fragment.

The term “antagonist” means any molecule that blocks, prevents, inhibits, or neutralizes the normal function of SIGLEC-6. One type of antagonist is a molecule that interferes with the interaction between SIGLEC-6 and its ligand, including, but not limited to, an antibody or antibody fragment. Another type of antagonist is an antisense nucleotide that inhibits proper transcription of native SIGLEC-6 or siRNA that binds to the native transcript.

The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F(ab′)2 fragments) which are capable of specifically binding to protein. Fab and F(ab′)2 fragments lack the Fc fragment of intact antibody, clear more rapidly from the circulation of the animal or plant, and may have less non-specific tissue binding than an intact antibody (Wahl et al., J. Nucl. Med. 24:316-325 (1983)). Thus, these fragments are preferred, as well as the products of a FAB or other immunoglobulin expression library. Moreover, antibodies of the present invention include chimeric, single chain, and humanized antibodies. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. An antibody may bind to a SIGLEC-6 protein with a binding affinity (Kd) of at least about 10⁻⁸, 10⁻⁹, 10⁻¹¹, 10⁻¹¹, 10⁻¹² M. Antibodies of the present invention also include single-domain antibodies in which the functional antibody comprises only a heavy chain or light chain, such as those described in WO04081026 and WO04041865.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

The term “antibody fragment” refers to a portion of a full-length antibody, generally the target binding or variable region. Examples of antibody fragments include Fab, Fab′, F(ab′)₂ and Fv fragments. The phrase “functional fragment” of an antibody is a compound having qualitative biological activity in common with a full-length antibody. For example, a functional fragment or analog of an anti-SIGLEC-6 antibody is one which can bind to SIGLEC-6 in such a manner so as to act like the natural ligand and activate the ITIM, thus inhibiting mast cell functions. As used herein, “functional fragment” with respect to antibodies, includes Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for an target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single targetic site. Furthermore, in contrast to conventional (polyclonal) antibody preparations which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using the well known techniques. The parent monoclonal antibodies to be used in accordance with the present invention may be made by the hybridoma method first described by Kohler and Milstein, Nature 256, 495 (1975), or may be made by recombinant methods.

As used herein, the term “SIGLEC-6-mediated disorder” means a condition or disease which is characterized by the loss of SIGLEC-6 inhibition and mast cell activation, proliferation, or histamine release. Specifically it would be construed to include conditions associated with anaphylactic hypersensitivity and atopic allergies, including for example: asthma, allergic rhinitis & conjunctivitis (hay fever), eczema, urticaria, atopic dermatitis, and food allergies. The serious physiological condition of anaphylactic shock caused by, e.g., bee stings, snake bites, food or medication, is also encompassed under the scope of this term.

As used herein, the term “B-cell related disorder” means a condition or disease which is characterized by B-cells expressing SIGLEC-6 and having abnormal expression, activation, or cytokine release. Specifically it would be construed to include conditions associated with pathological B-cell profiles, such as B-cell lymphomas.

A “biologically active fragment of SIGLEC-6” refers to a fragment of SIGLEC-6 that is sufficient for mediating at least one of its biological activities, such as degranulation. A biologically active fragment preferably comprises the ITIM domain, optionally the SLAM domain, a portion comprising both of these conserved domains, such as the intracellular domain. A biologically active fragment of SIGLEC-6 may also comprise all or a fragment of the extracellular domain and may also comprise the transmembrane domain.

This invention is not limited to the particular methodology, protocols, cell lines, vectors, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

The present invention may be understood more readily by reference to the following detailed description of the invention and the Examples included herein. SIGLEC-6 was found to be differentially expressed in human primary mast cells as compared with other cell types, SIGLEC-6 was also expressed in circulating blood monocytes (CBMCs). SIGLEC-6 may, therefore, play an important role in stimulating mast cell activities in airway, and/or peripheral or connective tissues for inflammatory and allergic responses.

Thus, SIGLEC-6 may be used as a therapeutic target for treating mast cell mediated diseases such as allergic and nonallergic asthma, chronic obstructive pulmonary disease (COPD), allergic rhinitis, anaphylaxis, allergic gastrointestinal disease, atopic dermatitis, rheumatoid arthritis, system sclerosis, and other allergic, autoimmune and inflammatory diseases. Activators/inhibitors of SIGLEC-6 for this treatment can be antibodies, peptide mimetics for the ligand, small molecules, antisense, or RNAi.

### Identification of Siglec-6

Genes differentially expressed in human mast cells were identified initially by comparing the mRNA expression levels among mast cells (cultured from umbilical cord blood CD34+ cells), PBMC peripheral blood mononuclear cells), and THP-1 (acute monocytic leukemia; lymphocytes) using the gene microarray technology. (See examples below.) The differential expression of SIGLEC-6 in mast cells was further confirmed by quantitative RT-PCR using a number of different cell types and humans tissues.

### Agonists and Antagonists

The present invention provides agonists and antagonists that directly or indirectly activate or inhibit the expression or action SIGLEC-6, or bind for purposes of detection. Types of agonist and antagonists include, but are not limited to, polypeptides, proteins, peptides, glycoproteins, glycopeptides, glycolipids, polysaccharides, oligosaccharides, nucleotides, organic molecules, bioorganic molecules, peptidomimetics, pharmacological agents and their metabolites, and transcriptional and translation control sequences.

In one embodiment, the agonist may be an antibody that binds efficiently with the extracellular domain of SEQ ID NO:2. These antibodies bind to SIGLEC-6 activating the ITIM, and hence inhibit activation of the mast cell and/or inhibit the B-cell alleviating B-cell related disorders.

The agonist may also include antibodies that bind specifically to SIGLEC-6 and influence biological actions and functions, e.g., to activate or inhibit the production of cytokines. Agonist antibodies can be polyclonal or monoclonal, and may be chimeric, human, humanized, or deimmunized.

Agonist antibodies may be used to prevent or treat diseases characterized by mast cell proliferation and/or degranulation. Agonists may be used for the treatment of various immune diseases, including, but not limited to allergic diseases such as asthma, allergic rhinitis, atopic dermatitis, food hypersensitivity and urticaria; transplantation associated diseases including graft rejection and graft-versus-host-disease; autoimmune or immune-mediated skin diseases including bullous skin diseases, erythema multiform and contact dermatitis, psoriasis; rheumatoid arthritis, juvenile chronic arthritis; inflammatory bowel disease (i.e., ulcerative colitis, Crohn's disease); systemic lupus erythematosis; spondyloarthropathies; systemic sclerosis (scleroderma); idiopathic inflammatory myopathies (dermatomyositis, polymyositis); Sjogren's syndrome; systemic vasculitis; sarcoidosis; autoimmune hemolytic anemia (immune pancytopenia, paroxysmal nocturnal hemoglobinuria), autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinatingpolyneuropathy or Guillain-Barre syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory and fibrotic lung diseases such as cystic fibrosis, gluten-sensitive enteropathy, and Whipple's disease; immunologic diseases of the lung such as eosinophilic pneumonia, idiopathic pulmonary fibrosis and hypersensitivity pneumonitis.

Antibodies of the present invention may also be used for diagnostic purposes to detect the presence and/or levels of mast cells by measuring SIGLEC-6 binding in a patient sample, such as a tissue biopsy or a sputum sample.

### Polypeptides

In another aspect, the present invention provides a method of treatment employing an isolated polypeptide of SIGLEC-6 having the amino acid sequence of SEQ ID NO:2; a variant of SEQ ID NO:2; or the extracellular domain fragment of SEQ ID NO:2. In one embodiment, the isolated polypeptide may be useful for blocking binding of the natural ligand to SIGLEC-6 on the surface of mast cells. In order to break tolerance, the peptide may be conjugated to any T-cell epitope, for example, tetanus toxin, diphtheria toxin or pertussis.

### Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody that specifically binds to SIGLEC-6, preferably, an antibody that binds to a polypeptide having the amino acid sequence of SEQ ID NO:2, particularly the extracellular domain of SEQ ID NO 2.

The polynucleotides may be obtained, and the nucleotide sequence of the polynucleotides determined, by commonly used nucleic acid sequencing methods known in the art. If the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

### Vectors

In another aspect, the present invention provides a vector comprising a nucleotide sequence encoding anti-SIGLEC-6 antibodies of the present invention and a host cell comprising such a vector. In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. Also, pGEX vectors may be used to express foreign polypeptides as fusion proteins of glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

In addition, sequences encoding appropriate signal peptides that are not naturally associated with SIGLEC-6 can be incorporated into expression vectors. For example, a nucleotide sequence for a signal peptide (secretory leader) may be fused in-frame to the polypeptide sequence so that the anti-SIGLEC-6 antibody is initially translated as a fusion protein comprising the signal peptide. A signal peptide that is functional in the intended host cells enhances extracellular secretion of the appropriate polypeptide. The signal peptide may be cleaved from the polypeptide upon secretion from the cell.

### Host Cells

Suitable host cells for expression of SIGLEC-6 and anti-SIGLEC-6 polypeptides include prokaryotes, yeast, and other eukaryotic cells. Prokaryotes useful as host cells in the present invention include gram negative or gram positive organisms such as E. coli or Bacilli. In a prokaryotic host cell, a polypeptide may include an N-terminal methionine residue to facilitate expression of the recombinant polypeptide in the prokaryotic host cell. The N-terminal Met may be cleaved from the expressed recombinant SIGLEC-6 receptor polypeptide. Promoter sequences commonly used for recombinant prokaryotic host cell expression vectors include β-lactamase and the lactose promoter system.

Yeasts useful as host cells in the present invention include those from the genus Saccharomyces, Pichia, K. Actinomycetes and Kluyveromyces. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes. Other suitable promoters and vectors for yeast and yeast transformation protocols are well known in the art.

Mammalian or insect host cell culture systems well known in the art may also be employed to express recombinant SIGLEC-6, e.g., Baculovirus systems for production of heterologous proteins in insect cells (Luckow and Summers, Bio/Technology 6:47 (1988)), or NSO or Chinese hamster ovary (CHO) cells for mammalian expression may be used. Transcriptional and translational control sequences for mammalian host cell expression vectors may be excised from viral genomes. Commonly used promoter sequences and enhancer sequences are derived from Polyonia virus, Adenovirus 2, Simian Virus 40 (SV40), and human cytomegalovirus.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

### Antibodies

The present invention provides an antibody that binds to SIGLEC-6 and methods for producing such an antibody, including antibodies that function as native SIGLEC-6 agonists, antagonists, or surface binders. Antibodies of the invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above.

In one embodiment, the method comprises using isolated epitope-bearing polypeptides of SIGLEC-6 or antigenic fragments thereof as an immunogen for producing antibodies that bind to the SIGLEC-6 in a known protocol for producing antibodies. Methods well known in the art include, but are not limited to, in vivo immunization, in vitro immunization, and phage display methods. See, e.g., Sutcliffe et al., supra; Wilson et al., supra, and Bittle et al., J. Gen. Virol., 66:2347-2354 (1985). Further, polypeptides of SIGLEC-6 may be used to generate antibodies which immunospecifically bind a polypeptide, polypeptide fragment, or variant of SEQ ID NO:2, and/or an epitope, as determined by immunoassays well known in the art for assaying specific antibody-antigen binding.

Antibodies may also be selected by panning a library of human scFv for those which bind SIGLEC-6 (Griffiths et. al., EMBO J. 12:725-734 (1993)). The specificity and activity of specific clones can be assessed using known assays (Griffiths et. al.; Clarkson et. al., Nature, 352: 642-648 (1991)). After a first panning step, one obtains a library of phage containing a plurality of different single chain antibodies displayed on phage having improved binding for SIGLEC-6. Subsequent panning steps provide additional libraries with higher binding affinities. Monovalent display can be accomplished with the use of phagemid and helper phage. Suitable phage include M13, fl and fd filamentous phage. Fusion protein display with virus coat proteins is also known and may be used in this invention.

To screen for antibodies which bind to a particular epitope on the antigen of interest (e.g., those which block binding of any of the antibodies disclosed herein to SIGLEC-6), a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al., J. Biol. Chem. 270:1388-1394 (1995), can be performed to determine whether the antibody binds an epitope of interest.

The antibodies may be human antigen-binding antibody fragments of the present invention and include, but are not limited to, Fab, Fab′ and F(ab′)2, Fd, single-chain Fvs (scFv), single-chain antibodies, disulfide-linked Fvs (sdfv) and fragments comprising either a VL or VH domain. Antigen-binding antibody fragments, including single-chain antibodies, may comprise the variable region(s) alone or in combination with the entirety or a portion of the following: hinge region, CH1, CH2, and C3 domains. Also included in the invention are antigen-binding fragments also comprising any combination of variable region(s) with a hinge region, CH1, CH2, and CH3 domains.

The antibodies of the invention may be from any animal origin including birds and mammals. Preferably, the antibodies are human, murine (e.g., mouse and rat), sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions or by size in contiguous amino acid residues.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention.

Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities may range in dissociation constant or Kd from 10⁻² M to 10⁻¹⁵ M.

Antibodies of the present invention may act as agonists, antagonists, or specific binders of SIGLEC-6. For example, the present invention includes antibodies which disrupt the receptor/ligand interactions between SIGLEC and its ligand either partially or fully. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). Further included in the invention are antibodies which activate the receptor. These antibodies may act as receptor agonists, i.e., potentiate or activate either all or a subset of the biological activities of the ligand-mediated receptor activation. The antibodies may be specified as agonists, antagonists or inverse agonists for biological activities comprising the specific biological activities. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 11 (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et a Cytokine 8(1):14-20 (1996) (which are all incorporated by reference herein in their entireties).

As discussed in more detail below, the antibodies of the present invention may be used either alone or in combination with other compounds or compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in diagnostic or detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, e.g., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting-blocking groups, proteolytic cleavage, linkage to a cellular ligand or other protein such as albumin, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Method of Making Antibodies to Siglec-6

Antibodies of the invention may be made by any method know. Typically, a SIGLEC-6 polypeptide fragment or protein may be administered to various host animals including, but not limited to, rabbits, mice, rats, etc. to induce the production of sera containing polyclonal antibodies specific for SIGLEC-6. The administration of a SIGLEC-6 polypeptide may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of SIGLEC-6 or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate).

The animal may be immunized using whole cells. Generally, either peripheral blood lymphocytes (“PBLs”) are used if cells of human origin are desired, or spleen cells or lymph node cells are used if non-human mammalian sources are desired. The host animal may also be immunized using an expression vector containing a cDNA encoding the desired immunogenic protein. The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2.sup.nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

In another embodiment, the antibodies of the present invention may also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.). Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988) and Presta, Curr. Op. Struct. Biol., 2:593-596 (1992).

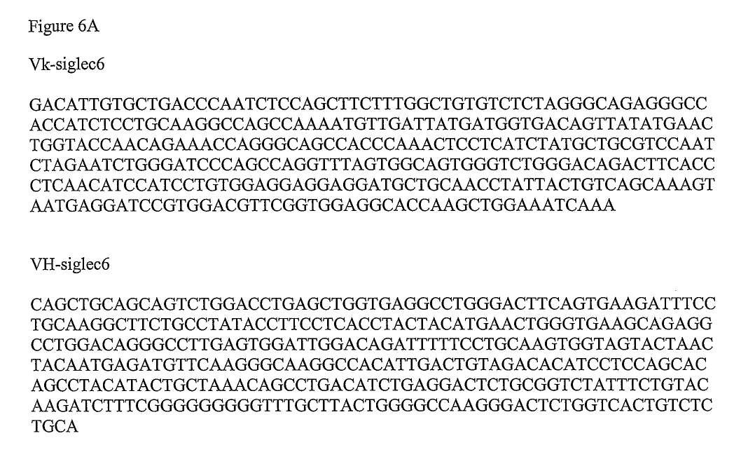

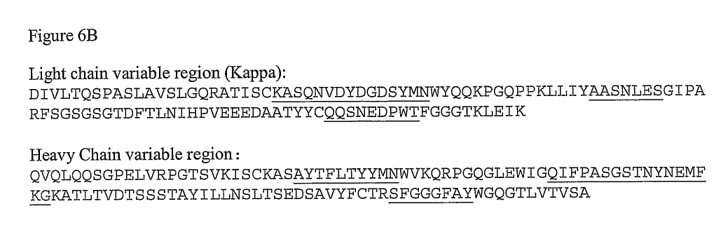

The nucleic acid (SEQ ID NOs: 7 and 9) and amino acid (SEQ ID NOs: 8 and 10) sequences of the variable regions of a preferred antibody of the invention are shown in FIGS. 6A and 6B. SEQ ID NOs: 7 and 8 are the nucleic acid and amino acid respectively of the light chain variable region, and SEQ ID NOs: 9 and 10 are the nucleic acid and amino acid respectively of the heavy chain variable region, with CDRs or Complementarity Determining Regions (hypervariable regions) underlined in these sequences.

Additional preferred antibodies of the invention will have substantial sequence identity to either one or both of the light chain or heavy sequences shown in FIGS. 6A and 6B. More particularly, preferred antibodies include those that have at least about 70 percent homology (sequence identity) to SEQ ID NOs: 8 and/or 10, more preferably about 80 percent or more homology to SEQ ID NOs: 8 and/or 10, still more preferably about 85, 90 or 95 percent or more homology to SEQ ID NOs: 8 and/or 10.

Preferred antibodies of the invention will have high sequence identity to CDR regions (underlined in FIG. 6b) of SEQ ID NOs: 8 and/or 10. Especially preferred antibodies of the invention will have one, two or three CDRs of a light chain variable region that have high sequence identity (at least 90% or 95% sequence identity) to or be the same as one, two or three of the corresponding CDRs of the light chain variable region of Mab 239-90 and are the following: 1) KASQNVDYDGDSYMN (SEQ ID NO: 12); 2) AASNLES (SEQ ID NO: 14); and 3) QQSNEDPWT (SEQ ID NO: 16)).

Especially preferred antibodies of the invention also will have one, two or three CDRs of a heavy chain variable region that have high sequence identity (at least 90% or 95% sequence identity) to or be the same as one, two or three of the corresponding CDRs of the heavy chain variable region of Mab 239-90 and are the following: 1) AYTFLTYYMN (SEQ ID NO: 18); 2) QIFPASGSTNYNEMFKG (SEQ ID NO: 20); and 3) SFGGGFAY (SEQ ID NO: 22).

Generally preferred nucleic acids of the invention will express an antibody of the invention that exhibits the preferred binding affinities and other properties as disclosed herein.

Preferred nucleic acids of the invention also will have substantial sequence identity to either one or both of the light chain or heavy sequences shown in FIG. 6A. More particularly, preferred nucleic acids will comprise a sequence that has at least about 70 percent homology (sequence identity) to SEQ ID NOs: 7 and/or 9, more preferably about 80 percent or more homology to SEQ ID NOs: 7 and/or 9, still more preferably about 85, 90 or 95 percent or more homology to SEQ ID NOs: 7 and/or 9.

Particularly preferred nucleic acid sequences of the invention will have high sequence identity to CDRs (shown with underlining in FIG. 6A) of SEQ ID NOs: 7 and/or 9. Especially preferred nucleic acids include those that code for an antibody light chain variable region and have one, two or three sequences that code for CDRs and have high sequence identity (at least 90% or 95% sequence identity) to or be the same as one, two or three of the sequences coding for corresponding CDRs of Mab 239-90 (those hypervariable regions shown with underlining in FIG. 6 and are the following: 1) aaggccagccaaaatgttgattatgatggtgacagttatatgaac (SEQ ID NO: 1); 2) gctgcgtccaatctagaatct (SEQ ID NO: 13); and 3) cagcaaagtaatgaggatccgtggacg (SEQ ID NO: 15)).

Especially preferred nucleic acids also code for an antibody heavy chain variable region and have one, two or three sequences that code for CDRs and have high sequence identity (at least 90% or 95% sequence identity) to or be the same as one, two or three of the sequences coding for corresponding CDRs of Mab 239-90 (those CDRs shown with underlining in FIG. 6 and are the following: 1) gcctataccttcctcacctactacatgaac (SEQ ID NO: 17); 2) cagatttttcctgcaagtggtagtactaactacaatgagatgttcaagggc (SEQ ID NO: 19); and 3) tctttegggggggggtttgcttac (SEQ ID NO: 21)).

Antibodies may also comprise one or more of the CDRs described herein in which one or more amino acids are substituted (e.g., with a conserved amino acid), deleted or added.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147(1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the SIGLEC-6 can, in turn, be utilized to generate anti-idiotype antibodies that “mimic” the receptor using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of SIGLEC-6 to a ligand can be used to generate anti-idiotypes that “mimic” the polypeptide multimerization and/or binding domain and, as a consequence, bind to and neutralize SIGLEC-6 and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The antibodies of the present invention may be bispecific antibodies. Bispecific antibodies are monoclonal, preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Methods of Producing Antibodies

The antibodies of the invention can be produced by any method known in the art for the generation or synthesis of antibodies, in particular, by chemical synthesis or by recombinant expression techniques.

Recombinant expression of an antibody of the invention, or fragment, derivative or analog thereof, (e.g., a heavy or light chain of an antibody of the invention or a single chain antibody of the invention), involves the construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341 (1992) (said references incorporated by reference in their entireties).

As discussed, supra, the polypeptides corresponding to a polypeptide, polypeptide fragment, or a variant of SEQ ID NO:2 may be fused or conjugated to the above antibody portions to increase the in vivo half life of the polypeptides or for use in immunoassays using methods known in the art. Further, the polypeptides corresponding to SEQ ID NO:2 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, ¹³¹I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, .beta.-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

The present invention also encompasses the creation of synthetic antibodies directed against the polypeptides of the present invention. One example of synthetic antibodies is described in Radrizzani, M., et al., Medicina, (Aires), 59(6):753-8, (1999)). Recently, a new class of synthetic antibodies has been described and are referred to as molecularly imprinted polymers (MIPs) (Semorex, Inc.). Antibodies, peptides, and enzymes are often used as molecular recognition elements in chemical and biological sensors. However, their lack of stability and signal transduction mechanisms limits their use as sensing devices. Molecularly imprinted polymers (MIPs) are capable of mimicking the function of biological receptors but with less stability constraints. Such polymers provide high sensitivity and selectivity while maintaining excellent thermal and mechanical stability. MIPs have the ability to bind to small molecules and to target molecules such as organics and proteins' with equal or greater potency than that of natural antibodies. These “super” MIPs have higher affinities for their target and thus require lower concentrations for efficacious binding.

During synthesis, the MIPs are imprinted so as to have complementary size, shape, charge and functional groups of the selected target by using the target molecule itself (such as a polypeptide, antibody, etc.), or a substance having a very similar structure, as its “print” or “template.” MIPs can be derivatized with the same reagents afforded to antibodies. For example, fluorescent ‘super’ MIPs can be coated onto beads or wells for use in highly sensitive separations or assays, or for use in high throughput screening of proteins.

Moreover, MIPs based upon the structure of the polypeptide(s) of the present invention may be useful in screening for compounds that bind to the polypeptide(s) of the invention. Such a MIP would serve the role of a synthetic “receptor” by minimicking the native architecture of the polypeptide. In fact, the ability of a MIP to serve the role of a synthetic receptor has already been demonstrated for the estrogen receptor (Ye, L., Yu, Y., Mosbach, K, Analyst., 126(6):760-5, (2001); Dickert, F, L., Hayden, O., Halikias, K, P, Analyst., 126(6):766-71, (2001)). A synthetic receptor may either be mimicked in its entirety (e.g., as the entire protein), or mimicked as a series of short peptides corresponding to the protein (Rachkov, A., Minoura, N, Biochim, Biophys, Acta., 1544(1-2):255-66, (2001)). Such a synthetic receptor MIPs may be employed in any one or more of the screening methods described elsewhere herein.

MIPs have also been shown to be useful in “sensing” the presence of its mimicked molecule (Cheng, Z., Wang, E., Yang, X, Biosens, Bioelectron., 16(3):179-85, (2001); Jenkins, A, L., Yin, R., Jensen, J. L, Analyst., 126(6):798-802, (2001); Jenkins, A, L., Yin, R., Jensen, J. L, Analyst., 126(6):798-802, (2001)). For example, a MIP designed using a polypeptide of the present invention may be used in assays designed to identify, and potentially quantitate, the level of said polypeptide in a sample. Such a MIP may be used as a substitute for any component described in the assays, or kits, provided herein (e.g., ELISA, etc.).

A number of methods may be employed to create MIPs to a specific receptor, ligand, polypeptide, peptide, organic molecule. Several preferred methods are described by Esteban et al in J. Anal, Chem., 370(7):795-802, (2001), which is hereby incorporated herein by reference in its entirety in addition to any references cited therein. Additional methods are known in the art and are encompassed by the present invention, such as for example, Hart, B, R., Shea, K, J. J. Am. Chem., Soc., 123(9):2072-3, (2001); and Quaglia, M., Chenon, K., Hall, A, J., De, Lorenzi, E., Sellergren, B, J. Am. Chem., Soc., 123(10):2146-54, (2001); which are hereby incorporated by reference in their entirety herein.

### Agonist Screening

In another aspect, the present invention provides a screening method for identifying SIGLEC-6 agonists. The screening method comprises exposing SIGLEC-6 to a potential SIGLEC-6 agonist and determining whether the potential agonist activates the ITIM, thereby inhibiting cytokine or histamine release from the mast cell. (See Example 11). If the potential agonist binds to SIGLEC-6, there is a strong presumption that the potential agonist will actually function as an agonist when administered in vivo to a patient. The SIGLEC-6 agonists identified using this method can be characterized as an agonist by exposing mast cells to the agonist and measuring cytokine or histamine release in comparison to cells activated in the absence of the agonist. Agonists will prevent degranulation and/or histamine release by activating the ITIM. Another method for screening comprises transfecting the cells with a reporter gene construct that contains SIGLEC-6. The potential agonist may be an organic compound or polypeptide, including antibodies.

High throughput screening methodologies are particularly envisioned for the detection of modulators of SIGLEC-6 described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptides (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Iy.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a SIGLEC-6 polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, st ligands) that bind to expressed, and preferably purified polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a SIGLEC-6 polypeptide or peptide to measure a biological binding or ligand binding activity, the source may be a whole cell lysate that can be prepared by successive freeze-thaw cycles (e.g., one to three) in the presence of standard protease inhibitors. The SIGLEC-6 polypeptide may be partially or completely purified by standard protein purification methods, e.g., affinity chromatography using specific antibody described infra, or by ligands specific for an epitope tag engineered into the recombinant SIGLEC-6 polypeptide molecule, also as described herein. Binding activity can then be measured as described.

Compounds which are identified according to the methods provided herein, and which modulate or regulate the biological activity or physiology of the SIGLEC-6 polypeptides according to the present invention are a preferred embodiment of this invention. It is contemplated that such modulatory compounds may be employed in treatment and therapeutic methods for treating a condition that is mediated by SIGLEC-6 polypeptides by administering to an individual in need of such treatment a therapeutically effective amount of the compound identified by the methods described herein.

In addition, the present invention provides methods for treating an individual in need of such treatment for a disease, disorder, or condition that is mediated by the SIGLEC-6 polypeptides of the invention, comprising administering to the individual a therapeutically effective amount of the SIGLEC-6-modulating compound identified by a method provided herein.

### Therapeutic Uses of Antibodies

The present invention is further directed to antibody-based therapies which involve administering antibodies of the invention to an animal, preferably a mammal, and most preferably a human, for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of Siglec-6 or a polypeptide of the invention, including, but not limited to, any one or more of the diseases, disorders, or conditions described herein. The treatment and/or prevention of diseases, disorders, or conditions associated with Siglec-6 or aberrant expression and/or activity of a polypeptide of the invention includes, but is not limited to, alleviating symptoms associated with those diseases, disorders or conditions, eliminating the population of B-cells expressing Siglec-6, or depleting B-cells expressing Siglec-6. Antibodies of the invention may be provided in pharmaceutically acceptable compositions as known in the art or as described herein.

A summary of the ways in which the antibodies of the present invention may be used therapeutically includes binding Siglec-6 or polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human or humanized antibodies, fragments derivatives, analogs, or nucleic acids, are administered to a human patient for therapy or prophylaxis.

It is preferred to use high affinity and/or potent in vivo inhibiting and/or neutralizing antibodies against Siglec-6 or polypeptides or polynucleotides of the present invention, fragments or regions thereof, for both immunoassays directed to and therapy of disorders related to Siglec-6 or polynucleotides or polypeptides, including fragments thereof, of the present invention. Such antibodies, fragments, or regions, will preferably have an affinity for Siglec-6 or polynucleotides or polypeptides of the invention, including fragments thereof.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the gene encoding an antibody directed against a polypeptide of the present invention, said polypeptide having the potential to elicit an allergic and/or immune response in an organism, and transforming the organism with said antibody gene such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or gene therapy applications of the present invention are provided elsewhere herein.

Alternatively, antibodies of the present invention could be produced in a plant (e.g., cloning the gene of the antibody directed against a polypeptide of the present invention, and transforming a plant with a suitable vector comprising said gene for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

### Antibody-Based Gene Therapy

In a specific embodiment, nucleic acids comprising sequences encoding antibodies or functional derivatives thereof, are administered for the prophylaxis, treatment, or inhibition of a mast-cell mediated disease or disorder, by way of gene therapy. Gene therapy refers to therapy performed by the administration to a subject of an expressed or expressible nucleic acid. In this embodiment of the invention, the nucleic acids produce their encoded antibody that mediates a therapeutic effect.

In another embodiment, nucleic acids comprising sequences encoding antibodies or functional derivatives thereof, are administered for the prophylaxis, treatment, or inhibition of a B-cell mediated disease or disorder, wherein the B-cell is expressing by way of gene therapy.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

According to the invention, the nucleic acid sequences encoding the antibody are part of an expression vector that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem . . . 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient. In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In one embodiment, the therapy would be localized to the lung of an asthmatic patient to reduce or eliminate mast cells that are causing airway remodeling and asthmatic symptoms through cytokine induction and release. Current techniques used for gene therapy in cystic fibrosis patients would be applicable to the present invention.

Diagnosis and Imaging with Antibodies

Labeled antibodies, and derivatives and analogs thereof, which specifically bind to SIGLEC-6 can be used for diagnostic purposes to detect, diagnose, or monitor diseases, disorders, and/or conditions associated with B-cells expressing Siglec-6 and/or associated with the aberrant expression and/or activity of SIGLEC-6. The method comprises (a) assaying the expression of SIGLEC-6 in cells or body fluid of an individual using one or more antibodies of the invention and (b) comparing the level of expression with a standard expression level, whereby an increase or decrease in the assayed expression level compared to the standard expression level is indicative of a B-cell mediated disease or condition and/or aberrant expression.

The invention provides a diagnostic assay for diagnosing a disorder, comprising (a) assaying the expression of SIGLEC-6 in cells or body fluid of an individual using one or more antibodies of the invention and (b) comparing the level of expression with a standard expression level, whereby an increase or decrease in the assayed expression level compared to the standard expression level is indicative of a particular disorder.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention includes the in vivo detection and diagnosis of a mast cell mediated disease or disorder in an animal, such as a human. In one embodiment, diagnosis comprises: a) administering (for example, parenterally, subcutaneously, or intraperitoneally) to a subject an effective amount of a labeled anti-SIGLEC-6 antibody which specifically binds to the surface of mast cells; b) waiting for a time interval following the administering for permitting the labeled antibody to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. The background level can be determined by various methods including, comparing the amount of labeled molecule detected to a standard value previously determined for a particular system.

Another aspect of the invention includes the in vivo detection and diagnosis of a B-cell mediated disease or disorder in an animal, such as a human. In one embodiment, diagnosis comprises: a) administering (for example, parenterally, subcutaneously, or intraperitoneally) to a subject an effective amount of a labeled anti-SIGLEC-6 antibody which specifically binds to the surface of mast cells; b) waiting for a time interval following the administering for permitting the labeled antibody to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with B-cells expressing SIGLEC-6. The background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of mast cells which contain the specific protein. In vivo imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled antibody can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the antibody is labeled with a fluorescent compound and is detected in the patient using a fluorescence responsive scanning instrument. In another embodiment, the antibody is labeled with a positron emitting metal and is detected in the patent using positron emission-tomography. In yet another embodiment, the antibody is labeled with a paramagnetic label and is detected in a patient using magnetic resonance imaging (MRI).

### Disease Predisposition Diagnostic

In another aspect, the present invention provides a method for diagnosing the predisposition of a patient to develop a B-cell mediated disease and/or diseases caused by the unregulated expression of cytokines. The invention is based upon the discovery that the presence of or increased amount of SIGLEC-6 receptor in certain patient cells, tissues, or body fluids may be indicative of a predisposition to certain B-cell mediated or immune diseases.

In one embodiment, the method comprises collecting a cell, tissue, or body fluid sample suspected of containing mast cells from a patient, analyzing the tissue or body fluid for the modulated expression of SIGLEC-6, and predicting the predisposition of the patient to certain immune diseases based upon the level of expression of SIGLEC-6 receptor in the tissue or body fluid.

In another embodiment, the method comprises collecting a cell, tissue, or body fluid sample suspected of containing SIGLEC-6 expressing B-cells from a patient, analyzing the tissue or body fluid for the modulated expression of SIGLEC-6, and predicting the predisposition of the patient to certain immune diseases based upon the level of expression of SIGLEC-6 receptor in the tissue or body fluid.

In another embodiment, the method comprises collecting a cell, tissue, or body fluid sample known to contain a defined level of SIGLEC-6 receptor from a patient, analyzing the tissue or body fluid for the amount of SIGLEC-6 receptor in the tissue, and predicting the predisposition of the patient to certain immune diseases based upon the change in the amount of SIGLEC-6 receptor in the tissue or body fluid compared to a defined or tested level established for normal cell, tissue, or bodily fluid. The defined level of SIGLEC-6 receptor may be a known amount based upon literature values or may be determined in advance by measuring the amount in normal cell, tissue, or body fluids. Specifically, determination of SIGLEC-6 receptor levels in certain tissues or body fluids permits specific and early, preferably before disease occurs, detection of immune diseases in the patient. Immune diseases that can be diagnosed using the present method include, but are not limited to, the immune diseases described herein. In the preferred embodiment, the tissue or body fluid is peripheral blood, peripheral blood leukocytes, biopsy tissues such as lung or skin biopsies, and synovial fluid and tissue.

Prophylaxis and Treatment of B-Cell and/or Siglec-6 Mediated Disease

In another aspect, the present invention provides a method for treating SIGLEC-6 mediated diseases in a mammal. The method comprises administering a disease treating amount of a SIGLEC-6 modulating compound, such as an anti-SIGLEC-6 agonist antibody to the mammal. The agonist antibody binds to the SIGLEC-6 receptor and regulates cytokine and/or cellular receptor expression to produce cytokine levels characteristic of non-disease states. SIGLEC-6 mediated diseases include allergy, asthma, autoimmune, or other inflammatory disease, as well as mastocytosis.

In another aspect, the present invention provides a method for treating B-cell mediated diseases in a mammal. The method comprises administering a disease treating amount of a SIGLEC-6 modulating compound, such as an anti-SIGLEC-6 agonist antibody or a small molecule that mimics the natural ligand for SIGLEC-6 to the mammal. The agonist antibody binds to the SIGLEC-6 receptor and regulates cytokine and/or cellular receptor expression to produce cytokine levels characteristic of non-disease states. B-cell mediated diseases include, but are not limited to, leukemia and B-cell lymphomas.

The antibody used in the prophylaxis and treatment of these diseases may also be engineered to comprise an effector function for killing mast cells and/or SIGLEC-6 expressing B-cells or be conjugated to a moeity, such as a cytotoxin or an apoptosis inducing molecule.

The dosages of SIGLEC-6 modulating compound vary according to the age, size, and character of the particular mammal and the disease. Skilled artisans can determine the dosages based upon these factors. The SIGLEC-6 modulating compound can be administered in treatment regimes consistent with the disease, e.g., a single or a few doses over one to several days to ameliorate a disease state or periodic doses over an extended time for prophylaxis of allergy or asthma.

The SIGLEC-6 modulating compound can be administered to the mammal in any acceptable manner including oral administration, by injection, using an implant, aerosol into the lungs and the like. Injections and implants permit precise control of the timing and dosage levels used for administration. The SIGLEC-6 modulating compound may be administered parenterally. As used herein parenteral administration means by intravenous, intramuscularly, or intraperitoneal injection, or by subcutaneous implant.

When administered by injection, the SIGLEC-6 modulating compound can be administered to the mammal in an injectable formulation containing any biocompatible agent and compatible carrier such as various vehicles, adjuvants, additives, and diluents.

### Therapeutic/Prophylactic Administration and Compositions

The invention provides methods of treatment, inhibition and prophylaxis by administration to a subject of an effective amount of a compound or composition of the invention, preferably an antibody. The subject is preferably an animal, including but not limited to animals such as cows, pigs, horses, chickens, cats, dogs, etc., and is preferably a mammal, and most preferably human.

Formulations and methods of administration that may be employed when the compound comprises a nucleic acid or an immunoglobulin are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc.

Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the compounds or compositions of the invention locally to the area in need of treatment; this may be achieved by, for example, and not by way of limitation, local infusion or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides compositions. Such compositions comprise a therapeutically effective amount of a compound, and an acceptable carrier. The term “carrier” refers to a diluent, adjuvant, excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is a preferred carrier when the composition is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration. Aqueous vehicles such as water having no nonvolatile pyrogens, sterile water, and bacteriostatic water are also suitable to form injectable solutions. In addition to these forms of water, several other aqueous vehicles can be used. These include isotonic injection compositions that can be sterilized such as sodium chloride, Ringer's, dextrose, dextrose and sodium chloride, and lactated Ringer's. Nonaqueous vehicles such as cottonseed oil, sesame oil, or peanut oil and esters such as isopropyl myristate may also be used as solvent systems for the compositions. Additionally, various additives which enhance the stability, sterility, and isotonicity of the composition including antimicrobial preservatives, antioxidants, chelating agents, and buffers can be added.

The amount of the compound of the invention which will be effective in the treatment, inhibition and prophylaxis of a B-cell mediated disease or disorder associated expressing SIGLEC-6 or a SIGLEC-6 mediated disease or disorder associated with aberrant expression and/or activity can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible.

### Siglec-6 Receptor Protein Purification

The antibodies of the present invention may also be used in a method for isolating and purifying SIGLEC-6 receptor protein from recombinant cell cultures, contaminants, and native environments. The method comprises exposing a composition containing SIGLEC-6 receptor protein and contaminants to an anti-SIGLEC-6 antibody capable of binding to the receptors, allowing the SIGLEC-6 receptor protein to bind to the antibody, separating the antibody-receptor complexes from the contaminants, and recovering the SIGLEC-6 receptor protein from the complexes.

Various purification methods known in the art may also be used, e.g., affinity purification methods that recover SIGLEC-6 receptor protein from recombinant cell culture or native sources. In this method, the antibodies against SIGLEC-6 are immobilized on a suitable support such a Sephadex resin or filter paper using methods well known in the art. The immobilized antibody then is contacted with a sample composition or solution containing the SIGLEC-6 receptor protein to be purified. The support is then washed with a suitable solvent capable of removing substantially all the material in the sample except the SIGLEC-6 receptor protein bound to the immobilized antibody. Finally, the support is washed with another suitable solvent that that removes the SIGLEC-6 receptor protein from the antibody.

### Knockout Animals

In another aspect, the present invention provides a knockout animal comprising a genome having a heterozygous or homozygous disruption in its endogenous SIGLEC-6 receptor gene that suppresses or prevents the expression of biologically functional SIGLEC-6 receptor proteins. Preferably, the knockout animal of the present invention has a homozygous disruption in its endogenous SIGLEC-6 receptor gene. Preferably, the knockout animal of the present invention is a mouse. The knockout animal can be made easily using techniques known to skilled artisans. Gene disruption can be accomplished in several ways including introduction of a stop codon into any part of the polypeptide coding sequence that results in a biologically inactive polypeptide, introduction of a mutation into a promoter or other regulatory sequence that suppresses or prevents polypeptide expression, insertion of an exogenous sequence into the gene that inactivates the gene, and deletion of sequences from the gene.

Several techniques are available to introduce specific DNA sequences into the mammalian germ line and to achieve stable transmission of these sequences (transgenes) to each subsequent generation. The most commonly used technique is direct microinjection of DNA into the pronucleus of fertilized oocytes. Mice or other animals derived from these oocytes will be, at a frequency of about 10 to 20%, the transgenic founders that through breeding will give rise to the different transgenic mouse lines. Methods for generating transgenic animals via embryo manipulation and microinjection, particularly animals such as mice, have become conventional in the art, e.g., U.S. Pat. Nos. 4,736,866, 4,870,009, and 4,873,191 and in Hogan, B., Manipulating the Mouse Embryo, (Cold Spring Harbor Laboratory Press, Cold Spring Harbor, N.Y., 1986). Similar methods are used for production of other transgenic animals.

Embryonic stem cell (“ES cell”) technology can be used to create knockout mice (and other animals) with specifically deleted genes. Totipotent embryonic stem cells, which can be cultured in vitro and genetically modified, are aggregated with or microinjected into mouse embryos to produce a chimeric mouse that can transmit this genetic modification to its offspring. Through directed breeding, a mouse can thus be obtained that lacks this gene. Several other methods are available for the production of genetically modified animals, e.g., the intracytoplasmic sperm injection technique (ICSI) can be used for transgenic mouse production. This method requires microinjecting the head of a spermatocyte into the cytoplasm of an unfertilized oocyte, provoking fertilization of the oocyte, and subsequent activation of the appropriate cellular divisions of a preimplantation embryo. The mouse embryos thus obtained are transferred to a pseudopregnant receptor female. The female will give birth to a litter of mice. In ICSI applied to transgenic mouse production, a sperm or spermatocyte heads suspension is incubated with a solution containing the desired DNA molecules (transgene). These interact with the sperm that, once microinjected, act as a carrier vehicle for the foreign DNA. Once inside the oocyte, the DNA is integrated into the genome, giving rise to a transgenic mouse. This method renders higher yields (above 80%) of transgenic mice than those obtained to date using traditional pronuclear microinjection protocols.

This invention can be further illustrated by the following examples, although it will be understood that these examples are included merely for purposes of illustration and are not intended to limit the scope of the invention unless otherwise specifically indicated.

### EXAMPLES

### Example 1

### Identification of Mast Cell-Differentially Expressed Siglec-6

The non-redundant human protein database IPI (Internation Protein Index) was searched for novel molecules containing: 1) at least one immunoglobulin (Ig) domain, 2) at least one immunoreceptor tyrosine-based inhibitory motif (ITIM), and 3) a transmembrane region. These features are shared by many signal activating receptors mediating immune system functions. A Hidden Markov Model (HMM)-based method was employed for the Ig-domain search. The HMM, which was built from an alignment of 113 confident Ig domains and calibrated using program HMMER, which was obtained from the Pfam (version 6.6) database.

To search for proteins containing an ITIM motif, a PROSITE-formatted motif profile was first constructed based on the common features of the ITIM motif, and software “seedtop” (NCBI) was used to perform the search.

A large-scale transmembrane region prediction for all the IPI proteins was carried out by using software TMHMM version 2.0 (http://www.cbs.dtu.dk/services/TMHMM/).

Siglec-6 cDNA sequence was found to meet all three criteria.

### Example 2

### Microarray Analysis

RNA samples were sent to Expression Analysis where microarray experiments to reverse Northerns were performed (B. Phimister, Nature Genetics supplement, 21:1, 1999). The cDNA sample was labeled and hybridized to the Human Genome U133 Plus 2.0 Array GeneChip. The initial data were received as black and white pixilated images for each hybridization, which were then transferred to and analyzed by Affymetrix 5.0 ArraySuite Software. This software employs statistical alogorithms to calculate a quantitative value (Signal Intensity) and a qualitative value (present or Absent) for each transcript on the array.

### Example 3

### Real-Time Quantitative PCR Analysis of Siglec-6 mRNA Expression

Two oligonucleotide primers: 5′ TGGAGCTGCCTCAAGTAGGG 3′ (SEQ ID NO 3) and 5′CGCGGCAGGTGAAATCTCCT 3′ (SEQ ID NO 4), were synthesized based on the SIGLEC-6 nucleotide sequences following selection using Primer Express 2.0 (Applied Biosystems, Inc.), and then used to monitor the expression of SIGLEC-6.

Real-time quantitative PCR was performed with the ABI Prism 7900 (Applied Biosystems, Inc.) sequence detection system, using SYBR Green reagents, according to the manufacture's instructions. Total RNAs were isolated to measure the level of SIGLEC-6 mRNA in the following cells: Daudi (a B lymphoblast cell line derived from Burkitt's lymphoma, ATCC No. CCL-213), THP-1 (a monocytic leukemia cell line, ATCC No. TIB202), HMC-1, (a mastoma cell line); peripheral blood mononuclear cells (PBMC); primary monocytes; primary B cells; primary neutrophils; in vitro cultured mast cells at week 8-9. The first strand cDNA from brain, heart, kidney, liver, lung, spleen, thymus and trachea were from BD Bioscience Clontech (Palo Alto, Calif.).

Equal amounts of each of the RNAs from the cells indicated above were used in a reverse transcription reaction to generate first strand cDNAs, which were used as templates in quantitative PCR reactions to obtain the threshold amplification cycle (Cₜ). The Cₜ was normalized using the control Cₜ from 18S RNAs to obtain ΔCₜ. To compare relative levels of gene expression of SIGLEC-6 in different cells and tissues, ΔΔCₜ values were calculated by using the lowest expression level as the base, which were then converted to the values of relative expression difference.

The quantitative RT-PCR analysis showed that SIGLEC-6 mRNA was expressed at very high levels in human mast cells, both in primary cell culture and a mastoma cell line. (Table 1).

TABLE 1
Expression profile of SIGLEC-6 mRNA assessed by quantitative RT-PCR

| Tissue/Cell | Ct | Relative expression |
|---|---|---|
| Brain | 33.0 | 7.9 |
| Heart | 32.1 | 13.7 |
| Kidney | 33.1 | 6.6 |
| Liver | 35.9 | 1.0 |
| Lung | 31.4 | 22.5 |
| Spleen | 29.1 | 105.7 |
| Thymus | 30.2 | 50.1 |
| Trachea | 29.8 | 68.5 |
| PBMC Culture (1) | 28.4 | 117.4 |
| PBMC Culture (2) | 28.7 | 96.6 |
| Neutrophil Cell Culture (1) | 33.6 | 3.3 |
| Neutrophil Cell Culture (2) | 33.0 | 6.1 |
| Mast Cell Culture (1) | 20.5 | 26354.1 |
| Mast Cell Culture (2) | 19.9 | 38402.0 |
| THP Cell Line | 28.5 | 100.0 |
| Daudi Cell Line | 34.6 | 1.6 |
| Monocyte Cell Culture | 33.3 | 4.9 |
| HPB-All T-Cell Line | 33.7 | 3.2 |
| HMC-1 Mast Cell Line | 20.3 | 31908.0 |

### Example 4

### Expression Constructs of Siglec-6

The coding sequence of SIGLEC-6 (SEQ ID NO: 1) was PCR-amplified by using two oligo primers from the Siglec-6 sequence:

| | |
|---|---|
| 5′ CACCATGCTACCGCTGCTGCTGA | (SEQ ID NO: 5) |
| 3′: TCACTTGTGTATCTTGATTTCTG | (SEQ ID NO: 6) |

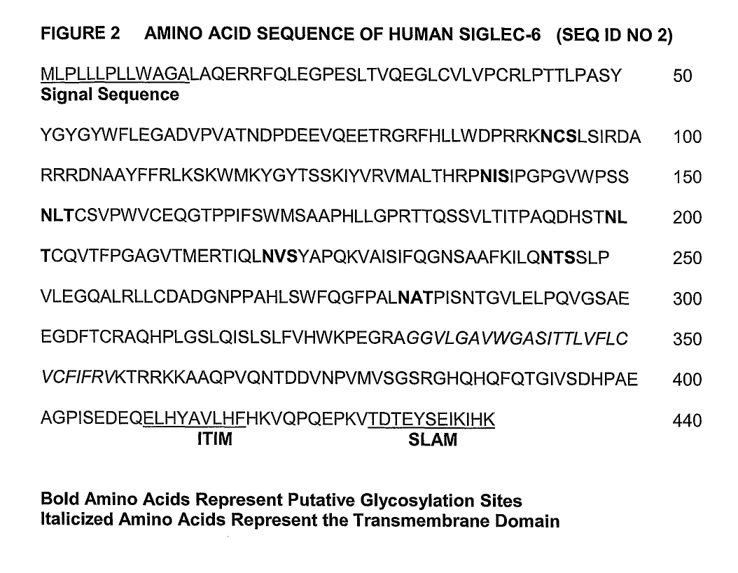

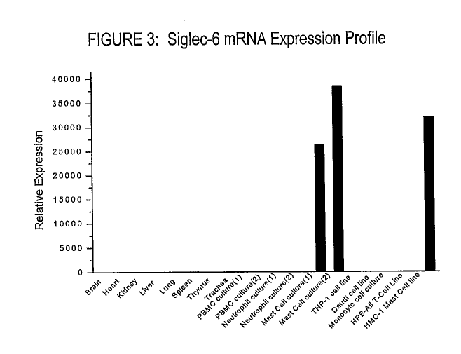

and cloned into pcDNA3.1D/V5-His vector (Invitrogen) with a V5 tag fused to the C-terminus. The resultant clone, pSiglec-6-V5, was transiently transfected into 293T cells. Forty-eight hours after transfection, transfected cells were harvested and separated into membrane and cytosolic fractions by either a homogenization or freeze-thaw method. Western blot analysis was performed using Anti-V5 MAb and anti-mouse IgG conjugates. SIGLEC-6 was expressed predominantly as a 55 kDa protein, which is larger than the calculated 50 kDa molecular weight, implying that SIGLEC-6 may be post-translationally modified, e.g., by glycosylation. Patel et al. (J. Biol. Chem., supra) reported seven possible glygosylation sites within the extracellular domain (See FIG. 2, Bold Sequences).

The sequence of SIGLEC-6 expression construct was verified to be identical to NM_{—}001245 (GenBank Accession Number).

### Example 5

### Diagnostic Assays for Siglec-6

To determine if SIGLEC-6 protein is expressed in cells, immunofluorescence experiments can be performed with whole blood, isolated peripheral blood mononuclear cells or in tissues, such as lymph nodes. Approximately 25,000 cells are cytospun onto glass slides and air-dried. Cells are fixed with Carnoy's Fix (60% ethanol, 30% chloroform and 10% acetic acid) for 10 minutes at room temperature, and washed with PBS three times. Cells are pre-blocked with block solution (1% horse serum, 2% rabbit serum, 1% BSA, and, 1% goat serum in PBS) on ice for 30 minutes and incubated with anti-SIGLEC-6 mAb (1 ug/ml in 1% BSA in PBS) for 30 minutes at room temperature. Cells are then washed three times and incubated with goat anti-mouse IgG (H+L)-FITC (Jackson Immuno Lab) at 1:100 dilution for 30 minutes at room temperature. Cells are washed, air dried and covered with coverslides. Fluorescence staining is examined using a fluorescence microscope and the results recorded using Snap-Shot software.

To perform FACS staining isolated cells are washed and pre-incubated at 4° C. for 30 minutes with blocking buffer (1% horse serum, 2% rabbit serum, 1% BSA, and, 1% goat serum in PBS). Cells are then incubated with FITC-conjugated anti-SIGLEC-6 mAb (10 μg/ml) or in the same buffer for 30 minutes. Alternatively, cells can be first stained with unconjugated anti-SIGLEC-6 mAb followed by FITC or PE conjugated anti-mouse IgG. After three washes, cells are fixed in 1×PBS with 1% paraformaldehyde. The samples are analyzed by FACScan (Becton Dickinson, Franklin Lakes, N.J.).

To perform immunohistochemistry, 10% formalin-fixed and paraffin-embedded or cryostat-acetone fixed serial sections of human tissues are used. Paraffin-embedded tissue samples are deparaffinized, rehydrated, incubated for 30 min at R.T. in PBS containing 2% normal goat serum, and then incubated overnight at 4° C. in buffer containing either 10 ug/ml of purified anti-SIGLEC-6 antibody or 0.5 ug/ml of mAb to a cell marker (Chemicon International, Temecula, Calif. clone #G3, MAB1222). Samples are washed, incubated for 1 h at room temperature in buffer containing AP-labeled goat anti-mouse IgG, washed twice in PBS, and incubated for 15 min at room temperature in alkaline phosphatase substrate solution (Pierce, Rockford, Ill.; Cat#34034). The antibody-stained tissue sections are counterstained with Gill's hematoxylin and covered with Immu-Mount (Shandon, Pittsburgh, Pa.).

### Example 6

### Immunohistochemistry

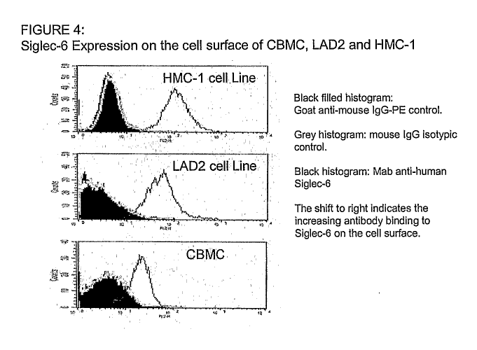

Fractionation of cells resulted in the presence of SIGLEC-6 in the membrane fraction, but very little was present in the cytosol. To further confirm SIGLEC-6 expression on the cell surface, three cell lines (CBMC, LAD2 and HMC-1) were immunostained with a PE-conjugated mouse anti-human SIGLEC-6 antibody. FACS analysis results are presented in FIG. 4, verifying that SIGLEC-6 is expressed on the cell surface of these cells.

In addition, mast cells in human lung and trachea tissue samples obtained from the Cooperative Human Tissue Network (CHTN), Southern division, university of Alabama at Birmingham were analyzed for the presence of SIGLEC-6.10% formalin-fixed and paraffin-embedded or cryostat-acetone fixed serial sections of human trachea and lung tissues were used. Each paraffin-embedded tissue specimen was deparaffinized, rehydrated, incubated for 30 min at R.T. in PBS containing 2% normal goat serum, and then incubated overnight at 4° C. in buffer containing either 10 ug/ml of purified anti-human SIGLEC-6 antibody (BD Pharmingen™ clone# E20-1232) or 0.5 ug/ml of anti-human tryptase antibody (Chemicon International, Temecula, Calif. clone #G3, MAB1222). Samples were washed, incubated for 1 h at room temperature in buffer containing AP-labeled goat anti-mouse IgG, washed twice in PBS, and incubated for 15 min at room temperature in alkaline phosphatase substrate solution (Pierce, Rockford, Ill.; Cat#34034). The antibody-stained tissue sections were counterstained with Gill's hematoxylin and covered with Immu-Mount (Shandon, Pittsburgh, Pa.).

Cells stained with anti-SIGLEC-6 also showed staining with anti-tryptase, an established cell marker for mast cells, revealing that SIGLEC-6 was expressed on mast cells in these tissue samples. The detection of SIGLEC-6 on tissue mast cells was consistent with the previous FACS results that indicated expression of this protein in cultured human mast cells as well as LAD2 cells.

### Example 7

### Anti-Siglec-6 Antibody Generation and Screening

Anti-SIGLEC-6 antibodies of the present invention may be generated by traditional hybridoma techniques well known in the art. Briefly, mice were immunized with SIGLEC-6 synthesized in vitro from the expression construct generated in Example 4 above. The immunogen was emulsified in complete Freund's adjuvant, and injected subcutaneously or intraperitoneally in amounts ranging from 10-100 μg. Ten to fifteen days later, the immunized animals are boosted with additional SIGLEC-6 emulsified in incomplete Freund's adjuvant. Mice were periodically boosted thereafter on a weekly to bi-weekly immunization schedule.

In addition, antibodies were also generated by administering the cDNA construct of Example 4 and the expression of SIGLEC-6 in vivo induced an immune response to the protein in the immunized mice.

Both types of mice were then used to generate hybridomas. For each fusion, single cell suspensions were prepared from the spleen of an immunized mouse and used for fusion with SP2/0 myeloma cells. SP2/0 cells (1×10⁸) and spleen cells (1×10⁸) were fused in a medium containing 50% polyethylene glycol (M.W. 1450) (Kodak, Rochester, N.Y.) and 5% dimethylsulfoxide (Sigma Chemical Co., St. Louis, Mo.). The cells were then adjusted to a concentration of 1.7×10⁵ spleen cells/ml of the suspension in DMEM medium (Gibco, Grand Island, N.Y.), supplemented with 5% fetal bovine serum and HAT (10 mM sodium hypoxanthine, 40 μM aminopterin, and 1.6 mM thymidine). Two hundred and fifty microliters of the cell suspension were added to each well of about fifty 96-well microtest plates. After about ten days culture supernatants were withdrawn for screening for reactivity with purified human SIGLEC-6 by ELISA.

Wells of Immulon II (Dynatech Laboratories, Chantilly, Va.) microtest plates were coated overnight with human SIGLEC-6 at 0.1 μg/ml (50 μl/well). The non-specific binding sites in the wells were then saturated by incubation with 200 μl of 5% BLOTTO (non-fat dry milk) in phosphate-buffered saline (PBS) for one hour. The wells were then washed with PBST buffer (PBS containing 0.05% TWEEN® 20). Fifty microliters of culture supernatant from each fusion well were added to the coated well together with 50 μl of BLOTTO for one hour at room temperature. The wells were washed with PBST. The bound antibodies were then detected by reaction with diluted horseradish peroxidase (HRP) conjugated goat anti-mouse IgG (Fc specific) (Jackson ImmunoResearch Laboratories, West Grove, Pa.) for one hour at room temperature. The wells were then washed with PBST. Peroxidase substrate solution containing 0.1% 3,3,5,5, tetramethyl benzidine (Sigma, St. Louis, Mo.) and 0.003% hydrogen peroxide (Sigma, St. Louis, Mo.) in 0.1M sodium acetate pH 6.0 was added to the wells for color development for 30 minutes. The reaction was terminated by addition of 50 μl of 2M H₂SO₄ per well. The optical density (OD) was read at 450 nm with an ELISA reader (Dynatech Laboratories, Chantilly, Va.).

Hybridomas in wells positive for SIGLEC-6 reactivity were single-cell cloned by a limiting dilution method. Monoclonal hybridomas were then expanded and culture supernatants collected for purification by protein A chromatography. The purified antibodies were then characterized for determination of affinity and kinetic binding constants by BIAcore and for effects on histamine release from mast cells.

Isolated monoclonal anti-Siglec-6 antibodies were sequenced following standard protocols used in the art. Nucleotide and amino acid sequences of light chain (kappa) and heavy chain (H) variable regions of monoclonal antibody Mab 239-90 are shown below with complementarity determining regions (CDRs) underlined:

### Light Chain Variable Region (Kappa):

(SEQ ID NO: 7)

gacattgtgctgacccaatctccagcttctttggctgtgtctctagggca
gagggccaccatctcctgcaaggccagccaaaatgttgattatgatggtg
acagttatatgaactggtaccaacagaaaccagggcagccacccaaactc
ctcatctatgctgcgtccaatctagaatctgggatcccagccaggtttag
tggcagtgggtctgggacagacttcaccctcaacatccatcctgtggagg
aggaggatgctgcaacctattactgtcagcaaagtaatgaggatccgtgg
acgttcggtggaggcaccaagctggaaatcaaa

(SEQ ID NO: 8)

DIVLTQSPASLAVSLGQRATISCKASQNVDYDGDSYMNWYQQKPGQPPKL
LIYAASNLESGIPARFSGSGSGTDFTLNIHPVEEEDAATYYCQQSNEDPW
TFGGGTKLEIK
Heavy Chain variable region:

(SEQ ID NO: 9)

cagctgcagcagtctggacctgagctggtgaggcctgggacttcagtgaa
gatttcctgcaaggcttctgcctataccttcctcacctactacatgaact
gggtgaagcagaggcctggacagggccttgagtggattggacagattttt
cctgcaagtggtagtactaactacaatgagatgttcaagggcaaggccac
attgactgtagacacatcctccagcacagcctacatactgctaaacagcc
tgacatctgaggactctgcggtctatttctgtacaagatctttcgggggg
gggtttgcttactggggccaagggactctggtcactgtctctgca

(SEQ ID NO: 10)

QVQLQQSGPELVRPGTSVKISCKASAYTFLTYYMNWVKQRPGQGLEWIGQ
IFPASGSTNYNEMFKGKATLTVDTSSSTAYILLNSLTSEDSAVYFCTRSF
GGGFAYWGQGTLVTVSA

### Underlined Regions Correspond to Kabat CDRs as Indicated Below for the Light Chain (Kappa):

| Vk-CDR1: | aaggccagccaaaatgttgattatgatggtgacagttatatgaac; | (SEQ ID NO: 11) |
|---|---|---|
| | KASQNVDYDGDSYMN; | (SEQ ID NO: 12) |
| Vk-CDR2: | gctgcgtccaatctagaatct; | (SEQ ID NO: 13) |
| | AASNLES; | (SEQ ID NO: 14) |
| Vk-CDR3: | cagcaaagtaatgaggatccgtggacg; | (SEQ ID NO: 15) |
| | QQSNEDPWT; | (SEQ ID NO: 16) |
| and for the heavy chain: | | |
| Vh-CDR1: | gcctataccttcctcacctactacatgaac | (SEQ ID NO: 17) |
| | AYTFLTYYMN; | (SEQ ID NO: 18) |
| Vh-CDR2: | cagatttttcctgcaagtggtagtactaactacaatgagatgttcaagggc | (SEQ ID NO: 19) |
| | QIFPASGSTNYNEMFKG; | (SEQ ID NO: 20) |
| Vh-CDR3: | tctttcgggggggggtttgcttac | (SEQ ID NO: 21) |
| | SFGGGFAY. | (SEQ ID NO: 22) |

### Example 8

### Effect of Siglec-6 on Mast Cell Degranulation

Human cord blood CD34⁺ cells (Bio-Whittaker, Walkersville, Md.) were cultured for 7 weeks in culture media consisting of RPMI1640 (Invitrogen) supplemented with 10% FBS (Sigma-Aldrich, St. Louis, Mo.), 2 mM L-glutamine, 50 μM 2-ME, 100 U/ml penicillin, 100 μg/ml streptomycin, 1 μg/ml gentamicin, 100 ng/ml SCF, 50 ng/ml IL-6 and 10 ng/ml IL-10. Cells were stained with anti-tryptase mAb to determine the percentage of mast cells. Cell suspensions were initially seeded at a density of 5×10⁵ cells/ml and cytokine-supplemented medium was replaced once a week.

The following protocol was used to activate human CBMC's with SIGLEC-6 via FcγRI cross-linking. The CBMC's, cultured as described above, were seeded into micotiter plates at 10,000 cells/100 ul/well. Cells were treated with 15 ng/ml of human rIFN-γ for 48 hours. Cells were then incubated for 1 hour at 37° C. with mouse anti-human FcγRI F(ab′)₂ in the presence of anti-SIGLEC-6 antibody (2 or 10 g/ml), or an irrelevant mast cell specific protein as a negative control. Plates were centrifuged at 1000 rpm for 2 minutes and the supernatant removed. 100 ul of fresh media was added followed by 10 μg/ml of goat anti-mouse IgG F(ab′)₂ and the plates were incubated for 2 hours. Supernatants were collected and the amount of histamine released due to cross-linking was measured.

The histamine release assay was carried out using standard reagents and protocols obtained from Beckman Coulter (Fullerton, Calif.). Briefly, the activated CBMC supernatants were harvested and their histamine contents measured using a histamine immunoassay kit (Beckman-Coulter, Palatine, Ill.) according to the manufacturer's protocol. The immunoassay was based on a competition between the histamine to be assayed and a histamine-alkaline phosphatase conjugate. The histamine present in the cell supernate was acylated with an acylating reagent at a slightly alkaline pH, and added onto microtiter wells coated with anti-histamine antibodies. Microtiter well were coated with a limited number of antibodies allowing for a competition to take place between the conjugate and the acylated histamine in the sample. After 2 hour of incubation at 4° C., the wells were rinsed to remove unbound components. Bound enzymatic activity was measured by adding a chromogenic substrate (pNPP). The color intensity was inversely proportional to the concentration of histamine in the sample. Histamine released was calculated on the basis of a standard curve obtained with standards provided in the kit.

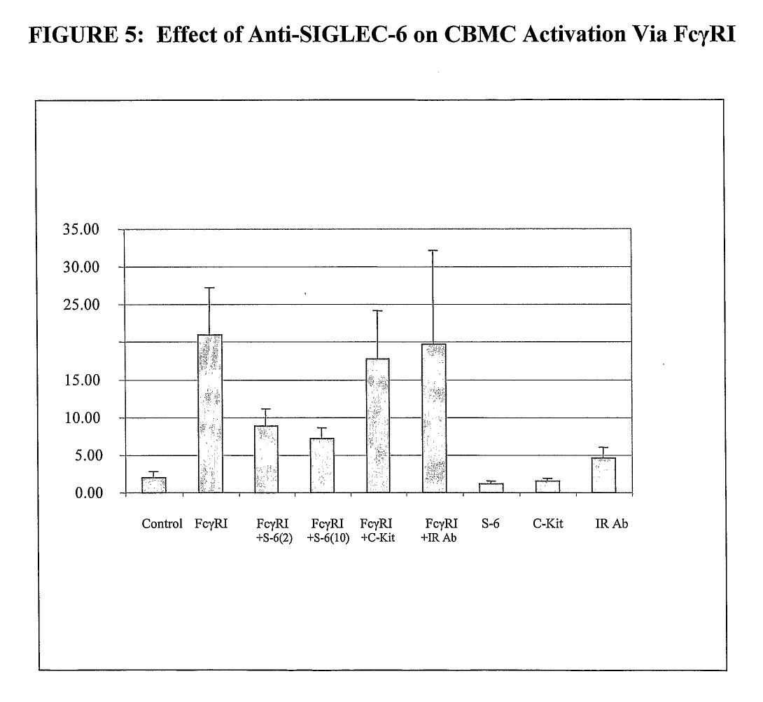

The results of this cross-linking experiment are presented in FIG. 5. If crosslinking occurred, then histamine will be released from the CBMC. As one observes, C-Kit (expressed on CBMCs) and an irrelevant antibody do not affect the amount of histamine released from the cells either in the presence or absence of FcγRI. In contrast, anti-SIGLEC-6 was able to inhibit the amount of histamine released in a dose dependent manner.

### Example 9

### ADCC Assay

An ADCC functional assay was established for screening anti-SIGLEC-6 monoclonal antibody candidates. Target cells (such as CBMC or LAD2) are labeled with 100 μCi ⁵¹Cr for 60 min and human PBMCs are used as the effector cells. The target cells (5×10³/well) and effector cells at various E:T ratios are coincubated in 200 μl of RPMI 1640 in a 96-well U-bottomed plate in triplicate for 6 h at 37° C. with either SIGLEC-6 (2 μg/well; Roche) or a control antibody. The radioactivity of the supernatant (100 μl) is then measured with a gamma counter. The percentage of specific lysis is calculated according to the formula: % specific lysis=100×(experimental cpm−spontaneous cpm)/(maximum cpm−spontaneous cpm). Controls include the incubation of target cells without antibody or an irrelevant antibody.

### Example 10

### Proliferation/Inhibition Assay

Anti-SIGLEC-6 monoclonal antibodies will be tested on mast cell line LAD2 cell to measure effects on cell proliferation and inhibition. H3-dCTP is added to the LAD2 cell culture medium in the presence of various concentration of anti-SIGLEC-6 antibody for a period of 7 days. The cells are washed to remove unincorporated H3-dCTP and the cells are fixed with 10% TCA before measuring the level of incorporation by scintillation counter. Increases in H3-dCTP incorporation indicate a proliferation effect by anti-SIGLEC-6 antibody on human mast cells, while decreases in H3-dCTP incorporation indicate an inhibitory effect of anti-SIGLEC-6 antibody on human mast cells.

### Example 11

### Mast Cell Apoptosis

Annexin V, a cellular marker of cell apoptosis, may be used to detect whether anti-SIGLEC-6 monoclonal antibodies have a biological effect on the process of mast cell apoptosis. FITC-conjugated anti-Annexin V will be used to do the FACS staining on human CBMC after 24 hours incubation with anti-SIGLEC-6 antibody candidates. Any cell surface Annexin V expression level will indicate an anti-SIGLEC-6 antibody effect on mast cell apoptosis.

### Example 12

### Agonist Screen

Since SIGLEC-6 molecules have two ITIM domains in the c-terminal bearing tyrosine site, any tyrosine phosphorylation by an antibody would be indicative of an agonist.

This experiment will be carried out by treating mast cells or a mast cell line, such as LAD2, with an anti-SIGLEC-6 monoclonal antibody candidate for five minutes in the normal cell culture condition, then lyse the cells, run the cell lysate in a western blot format, and probe the blot with monoclonal anti-phosphotyrosine. The antibodies candidates that cause SIGLEC-6 cytoplast domain protein tyrosine phosphorylation upon binding will be considered agonistic as compared to a similar cell culture grown in the absence of antibody.

1. (canceled)

2. An antibody, or fragment thereof, that binds SIGLEC-6, wherein the antibody comprises hypervariable regions having at least about 90% sequence identity to SEQ ID NOs: 12, 14, 16, 18, 20 or 22.

3. The antibody, or fragment thereof, of claim 2, wherein the light chain comprises a hypervariable region selected from the group consisting of Vk-CDR1 (SEQ ID NO: 12), Vk-CDR2 (SEQ ID NO: 14), and Vk-CDR3 (SEQ ID NO: 16).

4. The antibody, or fragment thereof, of claim 2, wherein the heavy chain comprises a hypervariable region selected from the group consisting of Vh-CDR1 (SEQ ID NO: 18), Vh-CDR2 (SEQ ID NO: 20), and Vh-CDR3 (SEQ ID NO: 22).

5. (canceled)

6. The antibody, or fragment thereof, of claim 2, wherein the light chain comprises Vk-CDR1 (SEQ ID NO: 12), Vk-CDR2 (SEQ ID NO: 14) and Vk-CDR3 (SEQ ID NO: 16).

7. (canceled)

8. The antibody, or fragment thereof, of claim 6, wherein the heavy chain comprises Vh-CDR1 (SEQ ID NO: 18), Vh-CDR2 (SEQ ID NO: 20), and Vh-CDR3 (SEQ ID NO: 22).

9. The antibody, or fragment thereof, of claim 2, wherein the heavy and light chains comprise a human framework region.

10. The antibody or fragment thereof of claim 8, wherein the light chain comprises the light chain variable region set forth in SEQ ID NO: 8.

11. The antibody or fragment thereof of claim 8, wherein the heavy chain comprises the heavy chain variable region set forth in SEQ ID NO: 10.

12. The antibody or fragment thereof of claim 10, wherein the heavy chain comprises the heavy chain variable region set forth in SEQ ID NO: 10.

13. The antibody of claim 9 further comprising a constant region.

14. (canceled)

15. A variable light and/or heavy chain of the antibody or fragment thereof of claim 2.

16. An isolated nucleic acid encoding the variable light and/or heavy chain of claim 15.

17. A composition comprising the isolated nucleic acid of claim 16.

18. A cell comprising an isolated nucleic acid of claim 16.

19. An antibody or fragment thereof that specifically binds to the same epitope in SIGLEC-6 as an antibody comprising a light chain having the CDRs of SEQ ID NOs 12, 14, 16, and a heavy chain having the CDRs of SEQ ID NOs 18, 20 and 22.

20. The antibody of claim 2, wherein the antibody is a human, a humanized, a chimeric, a single-chain, or a single-domain antibody.

21. A composition comprising the antibody of claim 2 and a suitable carrier, adjuvants, diluent, excipients, and/or additive.

22. A method of making a heavy and/or light chain or a fragment thereof, comprising culturing a cell of claim 18 under conditions appropriate for expression of the heavy and/or light chain, and purifying the heavy and/or light chain from the cell culture.

23. (canceled)

24. (canceled)

25. A method of prophylaxis and/or treatment of a mast-cell mediated disease or disorder in a subject, comprising administering to a subject in need thereof a SIGLEC-6 receptor modulator.

26. The method of claim 25, wherein the SIGLEC-6 receptor modulator is a SIGLEC-6 agonist.

27. The method of claim 25, wherein the SIGLEC-6 receptor modulator is a SIGLEC-6 antagonist.

28. The method of claim 25, wherein the SIGLEC-6 receptor modulator is an antibody.

29. A method of prophylaxis and/or treatment of a mast cell mediated disease or disorder in a subject, comprising administering to a subject in need thereof an antibody or fragment thereof of claim 2.

30. The method of claim 29, wherein the mast cell mediated disease or disorder is asthma.

31. A method for prophylaxis and/or treatment of a mast cell mediated disease or disorder in a subject, comprising administering to a subject in need thereof an anti-SIGLEC-6 antibody or fragment thereof having an effector function for killing mast cells.

32. The method of claim 31, wherein the effector function is antibody-dependent cell-mediated cytotoxicity (ADCC).

33. (canceled)

34. A method for prophylaxis and/or treatment of mast cell mediated disease comprising administering an anti-SIGLEC-6 antibody or fragment thereof conjugated to an apoptosis-inducing moiety for inducing apoptosis in mast cells.

35. The method of claim 34, wherein the apoptosis-inducing moiety is a pro-apoptotic member of the Bcl-2 family selected from Bax-α, Bak, Bcl-X_{S}, Bad, Bid, Bik, Erk, and Bok.

36. A method of prophylaxis and/or treatment of a B-cell mediated disease or disorder in a subject, comprising administering to a subject in need thereof an antibody or fragment thereof of claim 2.

37. A method for determining the presence of SIGLEC-6 in a biological sample, comprising contacting a biological sample with an antibody or fragment thereof of claim 2 and determining the presence of the antibody or fragment thereof.

38. A method of diagnosing a mast cell mediated disorder in a mammal comprising:
Obtaining a sample from a mammal suspected of having a mast cell mediated disorder;
Incubating said sample with a detectable amount of anti-SIGLEC-6 antibody;
Measuring the amount of bound antibody;
Comparing the amount of bound antibody in the suspected sample as compared to a normal control, wherein a different amount of bound antibody in the sample from the mammal suspected of having a mast cell mediated disorder relative to a normal control indicates that the mammal has or is likely to develop a mast cell mediated disorder.

39. A kit for detecting the presence of SIGLEC-6, comprising an antibody of claim 2.

40. A kit for diagnosing a SIGLEC-6 mediated disease or disorder comprising an anti-SIGLEC-6 antibody of claim 2.

41. A method of screening for an compound that modulates SIGLEC-6 receptor activity comprising:
preparing a cell comprising a nucleic acid sequence that encodes the amino acid sequence of SEQ ID NO 2 or a biologically active fragment thereof;
contacting said cell(s) with at least one compound whose ability to modulate the SIGLEC-6 receptor activity is sought to be determined;
monitoring said cell for modulation of the SIGLEC-6 receptor activity.

42. The method according to claim 41, wherein the cell is stably transfected.

43. The method according to claim 41, wherein the cell is transiently transfected.

44. The method according to claim 41, wherein the cell is a mast cell.

45. The method according to claim 41, wherein the compound is an agonist.

46. The method according to claim 41, wherein the compound is an antagonist.

47. The method according to claim 41, wherein the compound is an antibody.

48. The method according to claim 41, wherein the cells employed in step (a) further comprise a DNA encoding a reporter protein wherein said DNA is operatively linked to a SIGLEC-6 responsive transcription element.

49. The method according to claim 41, wherein step (b) is carried out in the presence of increasing concentrations of at least one compound whose ability to inhibit signal transduction activity of said receptor protein(s) is sought to be determined.

50. The method according to claim 48, wherein step (c) comprises monitoring in said cells the level of expression of the reporter protein as a function of the concentration of the compound, thereby indicating the ability of said compound to inhibit signal transduction activity.

51. A method of screening for agonists or antagonists of SIGLEC-6 activity comprising:
contacting cells that express SIGLEC-6 with a candidate compound;
assaying a cellular response; and
comparing the cellular response to a standard cellular response made in absence of the candidate compound; whereby, an increased cellular response over the standard indicates that the compound is an agonist and a decreased cellular response over the standard indicates that the compound is an antagonist.

52. A method of identifying a candidate compound for treatment of mast cell mediated inflammatory disorders in mammals comprising:
applying said compound to mast cells,
measuring expression/function of SIGLEC-6,
identifying whether said compound decreases said expression/function of SIGLEC-6 involved in mast cell mediated inflammatory disorders in comparison to mast cells to which said compound is not applied, and
identifying an expression/function-decreasing compound as a candidate compound for treatment of mast cell mediated inflammatory disorders.

53. The method of claim 52, wherein the candidate compound is a SIGLEC-6 agonist.

54. The method of claim 52, wherein the candidate compound is a SIGLEC-6 antagonist.

55. The method of claim 52, wherein the candidate compound is an antibody.

56. A method for identifying an agent that inhibits a biological activity of SIGLEC-6, comprising
contacting a polypeptide comprising the ITIM domain of SIGLEC-6 with an agent;
determining whether the agent binds to the ITIM domain of SIGLEC-6; and
determining whether an agent identified in b. as binding to the ITIM domain inhibits histamine release of activated mast cells.

57. The method of claim 56, wherein determining the biological activity of the SIGLEC-6 protein comprises determining the level of histamine release, wherein the release of a higher level of histamine release from the mast cell in the presence of the test agent relative to the absence of test agent indicates that the test agent is an agent that stimulates the biological activity of SIGLEC-6, whereas a lower level of histamine release from the mast cell in the presence of the test agent relative to the absence of test agent indicates that the test agent is an agent that inhibits the biological activity of SIGLEC-6.

58. A method for diagnosing a B-cell related disorder, wherein the B-cell expresses SIGLEC-6, in a mammal comprising:
Obtaining a sample from a mammal suspected of having a B-cell related disorder;
Incubating said sample with a detectable amount of anti-SIGLEC-6 antibody;
Measuring the amount of bound antibody; and
Comparing the amount of bound antibody in the suspected sample as compared to a normal control.

59. The method of claim 58, wherein the antibody is labeled.

60. The method of claim 59, wherein the label is a fluorescent moiety, a radioactivity moiety, an enzyme, or a luminescent moiety.

61. The method of claim 58, wherein the anti-SIGLEC-6 antibody is detected by a second antibody.

62. The method of claim 58, wherein the anti-SIGLEC-6 antibody is detected by ELISA.

63. A kit for diagnosing a B-cell related disorder comprising an anti-SIGLEC-6 antibody.

64. A kit according to claim 63, wherein the antibody is bound to a solid surface.

65. A method for the prophylaxis and/or treatment of a B-cell related disorder, wherein the B-cell expresses SIGLEC-6, in a patient comprising administering a SIGLEC-6 receptor modulator to a patient in need of such treatment.

66. The method of claim 65, wherein the SIGLEC-6 receptor modulator is a SIGLEC-6 agonist.

67. The method of claim 66, wherein the SIGLEC-6 receptor modulator is an antibody.

68. The method of claim 65, wherein the B-cell related disorder is B-cell lymphoma.

69. The method of claim 65, wherein the SIGLEC-6 receptor modulator is an anti-SIGLEC-6 antibody having an effector function for killing B-cells expressing SIGLEC-6.

70. The method of claim 69, wherein the effector function is antibody-dependent cell-mediated cytotoxicity (ADCC).

71. The method of claim 65, wherein the SIGLEC-6 receptor modulator is an anti-SIGLEC-6 antibody conjugated to an apoptosis-inducing moiety for inducing apoptosis in B-cells expressing SIGLEC-6.

72. The method of claim 71, wherein the apoptosis-inducing moeity is a pro-apoptotic member of the Bcl-2 family selected from Bax-α, Bak, Bcl-X_{S}, Bad, Bid, Bik, Erk, and Bok.

73. (canceled)

74. (canceled)

75. The antibody of claim 2, further comprising an apoptosis-inducing moiety.

76. The antibody of claim 75, wherein the apoptosis-inducing moiety is a pro-apoptotic member of the Bcl-2 family selected from Bax-α, Bak, Bcl-X_{S}, Bad, Bid, Bik, Erk, and Bok.
